(19) United States
(12) Patent Application Publication
(10) Pub. No.: US 2016/0029938 A1
(43) Pub. Date: Feb. 4, 2016

(54) DIAGNOSIS SUPPORTING DEVICE, DIAGNOSIS SUPPORTING METHOD, AND COMPUTER-READABLE RECORDING MEDIUM

(71) Applicant: JVC KENWOOD CORPORATION, Yokohama-shi (JP)

(72) Inventor: Katsuyuki SHUDO, Yokohama-shi (JP)

(21) Appl. No.: 14/805,050

(22) Filed: Jul. 21, 2015

(30) Foreign Application Priority Data
Jul. 31, 2014 (JP) 2014-156812
Jul. 31, 2014 (JP) 2014-156872
Nov. 28, 2014 (JP) 2014-242033

(51) Int. Cl. A61B 5/16; A61B 3/107; A61B 3/113; A61B 3/14; A61B 5/00

(52) U.S. Cl. CPC A61B 5/162; A61B 3/145; A61B 5/742; A61B 3/113; A61B 3/107; A61B 2576/00

(57) ABSTRACT

A diagnosis supporting device includes a display, an imaging unit that images a subject, a visual line detector that detects a visual line direction of the subject from an image imaged by the imaging unit, a visual point detector that detects a visual point of the subject in a display area of the display based on the visual line direction, an output controller that displays a diagnostic image representing a cause of a certain event and the event on the display, and an evaluator that calculates an evaluation value of the subject based on the visual point detected by the visual point detector when the diagnostic image is displayed.

### CROSS-REFERENCE TO RELATED APPLICATION(S)

The present application claims priority to and incorporates by reference the entire contents of Japanese Patent Application No. 2014-156812 filed in Japan on Jul. 31, 2014, Japanese Patent Application No. 2014-156872 filed in Japan on Jul. 31, 2014 and Japanese Patent Application No. 2014-242033 filed in Japan on Nov. 28, 2014.

### BACKGROUND OF THE INVENTION

1. Field of the Invention

The present invention relates to a diagnosis supporting device, a diagnosis supporting method and a computer-readable recording medium.

2. Description of the Related Art

It is often the case that developmentally disabled people have characteristics of deviated gazing points or difficulties in understanding causal relations. It's important in providing care and education (training) on developmentally disabled people to understand what point they gaze at to obtain information in order to understand a causal relation, whether they cannot understand the causal relation despite gazing at, or the like. Conventional techniques are described in Japanese Patent Application Laid-open No. 2011-206542 and Pierce K et al., “Preference for Geometric Patterns Early in Life as a Risk Factor for Autism,” Arch Gen Psychiatry. 2011 January; 68 (1): 101-109, for example.

However, the conventional methods cannot give an understanding of what point developmentally disabled people gaze at to obtain information in order to understand a causal relation, whether they cannot understand the causal relation despite gazing at, or the like. For this reason, the conventional methods may fail to appropriately support diagnosis, and a diagnosis supporting method with higher precision has been demanded.

### SUMMARY OF THE INVENTION

It is an object of the present invention to at least partially solve the problems in the conventional technology.

There is provided a diagnosis supporting device that includes a display, an imaging unit that images a subject, a visual line detector that detects a visual line direction of the subject from an image imaged by the imaging unit, a visual point detector that detects a visual point of the subject in a display area of the display based on the visual line direction, an output controller that displays a diagnostic image representing a cause of a certain event and the event on the display, and an evaluator that calculates an evaluation value of the subject based on the visual point detected by the visual point detector when the diagnostic image is displayed.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram illustrating an example of an arrangement of a display, a stereo camera, an infrared light source of an embodiment;

FIG. 2 is a diagram illustrating an example of the arrangement of the display, the stereo camera, the infrared light source, and a subject of the present embodiment;

FIG. 3 is a diagram illustrating an outline of functions of a diagnosis supporting device;

FIG. 4 is a block diagram illustrating an example of detailed functions of the units illustrated in FIG. 3;

FIG. 5 is a diagram illustrating an outline of processing performed by a diagnosis supporting device of the present embodiment;

FIG. 6 is an illustrative diagram illustrating a difference between a method of using two light sources and the present embodiment of using one light source;

FIG. 7 is a diagram for illustrating calculation processing for calculating the distance between a pupil center position and a corneal curvature center position;

FIG. 8 is a flowchart illustrating an example of calculation processing of the present embodiment;

FIG. 9 is a diagram illustrating a method for calculating the position of a corneal curvature center using a distance determined in advance;

FIG. 10 is a flowchart illustrating an example of visual line detection processing of the present embodiment;

FIG. 11 is a diagram for illustrating calculation processing of Modification 1;

FIG. 12 is a flowchart illustrating an example of the calculation processing of Modification 1;

FIG. 13 is a diagram illustrating an example of a diagnostic image used in the present embodiment;

FIG. 14 is a diagram illustrating an example of the diagnostic image used in the present embodiment;

FIG. 15 is a diagram illustrating an example of the diagnostic image used in the present embodiment;

FIG. 16 is a diagram illustrating an example of the diagnostic image used in the present embodiment;

FIG. 17 is a flowchart illustrating an example of diagnosis support processing of the present embodiment;

FIG. 18 is a diagram illustrating an example of a selection screen for selecting a primary answer;

FIG. 19 is a diagram illustrating an example of a right answer screen for displaying a right answer;

FIG. 20 is a diagram illustrating an example of an explanation screen;

FIG. 21 is a flowchart illustrating an example of gazing point detection processing;

FIG. 22 is a flowchart illustrating an example of analysis processing;

FIG. 23 is a diagram illustrating examples of a causal relation;

FIG. 24 is a flowchart illustrating an example of verification processing that verifies and displays effects of care and education;

FIG. 25 is a diagram for illustrating an example of a method for determining changes in measurement data;

FIG. 26 is a flowchart illustrating an example of analysis processing when a moving image is displayed;

FIG. 27 is a flowchart illustrating an example of training support processing of Modification 2;

FIG. 28 is a diagram illustrating an example of a menu screen of Modification 2;

FIG. 29 is a diagram illustrating an example of a still image 1 displayed in Modification 2;

FIG. 30 is a diagram illustrating an example of a still image 2 displayed in Modification 2;

FIG. 31 is a diagram illustrating an example of a selection screen of Modification 2;

FIG. 32 is a diagram illustrating an example of a right answer screen of Modification 2;

FIG. 33 is a diagram illustrating an example of an explanation screen of Modification 2;

FIG. 34 is a diagram illustrating an example of a reproduction screen of Modification 2; and

FIG. 35 is a diagram illustrating an example of implementing a training supporting device by a notebook PC.

### DETAILED DESCRIPTION OF THE PREFERRED EMBODIMENTS

The following describes embodiments of a diagnosis supporting device, a diagnosis supporting method, a computer-readable recording medium for supporting diagnosis according to the present invention in detail with reference to the drawings. The present invention is not limited by the embodiments. Although the following describes a case in which the diagnosis supporting device is used as the diagnosis supporting device that supports diagnosis of a developmental disorder or the like using a visual line detection result and can also be used for training, applicable devices are not limited to this.

The diagnosis supporting device of the present embodiment displays an image (video) indicating before and after an event, measures a dwell time at a position of a gazing point, and performs evaluation computation. A continuous moving image containing a scene representing a cause and a scene indicating before and after the event is displayed as an explanation image indicating a causal relation between the cause and the event. With this configuration, efficient training support that is easily understood by trainees is achieved.

The diagnosis supporting device of the present embodiment detects a visual line using an illuminator placed at one position. The diagnosis supporting device is not limited to the above embodiment. The diagnosis supporting device of the present embodiment, using a result measured by causing a subject to gaze at one point before visual line detection, calculates a corneal curvature center position with high accuracy.

The illuminator includes a light source and is a component that can apply light to an eyeball of the subject. The light source is, for example, an element that emits light such as a light emitting diode (LED). The light source may include one LED or include a plurality of LEDs combined and arranged at one position. The following may use a “light source” as a term thus representing the illuminator.

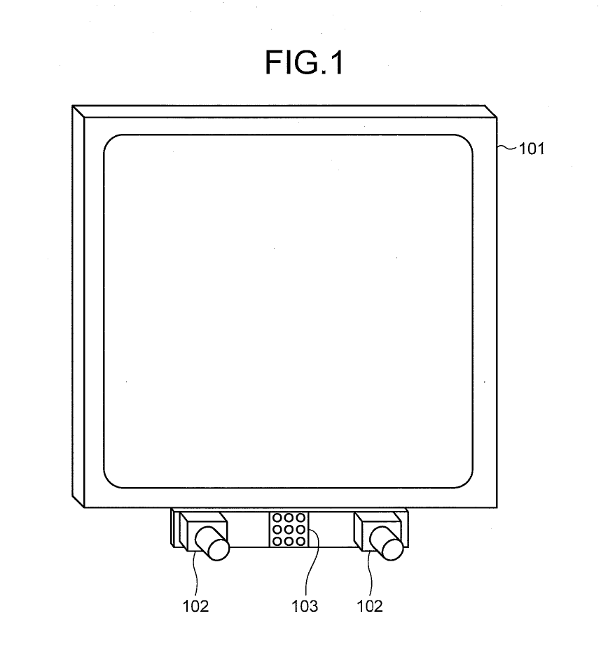

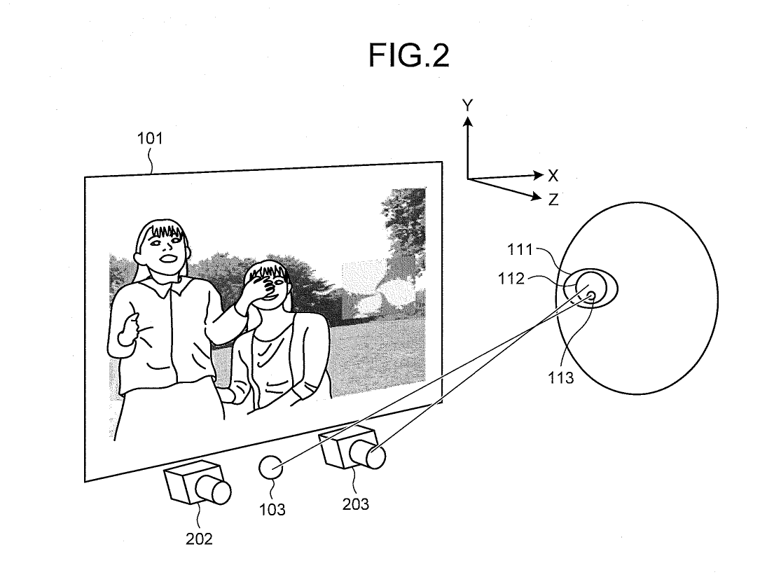

FIGS. 1 and 2 are diagrams illustrating an example of an arrangement of a display, a stereo camera, an infrared light source, and a subject of the present embodiment.

As illustrated in FIG. 1, this diagnosis supporting device 100 of the present embodiment includes a display 101, a stereo camera 102 corresponding to an imaging unit, and an LED light source 103. The stereo camera 102 is arranged below the display 101. The LED light source 103 is arranged at the central position between two cameras included in the stereo camera 102. The LED light source 103 is a light source that emits near-infrared rays with a wavelength of, for example, 850 nm. FIG. 1 illustrates an example of constituting the LED light source 103 (the illuminator) by nine LEDs. The stereo camera 102 uses lenses that can transmit near-infrared rays with a wavelength of 850 nm therethrough.

As illustrated in FIG. 2, the stereo camera 102 includes a right camera 202 and a left camera 203. The LED light source 103 applies near-infrared rays toward an eyeball 111 of the subject. In an image captured by the stereo camera 102, a pupil 112 reflects with low brightness and appears dark, whereas a corneal reflex 113 occurring as a virtual image within the eyeball 111 reflects with high brightness and appears bright. Positions of the pupil 112 and the corneal reflex 113 on the image can therefore be acquired by the two cameras (the right camera 202 and the left camera 203) separately.

From the positions of the pupil 112 and the corneal reflex 113 obtained by the two cameras, three-dimensional world coordinate values of the positions of the pupil 112 and the corneal reflex 113 are calculated. In the present embodiment, as three-dimensional world coordinates, with the central position of a screen of the display 101 as the point of origin, the up-and-down direction is a Y coordinate (the upper side is +), the lateral direction is an X coordinate (the right side viewed from the front is +), and the depth direction is a Z coordinate (the near side is +).

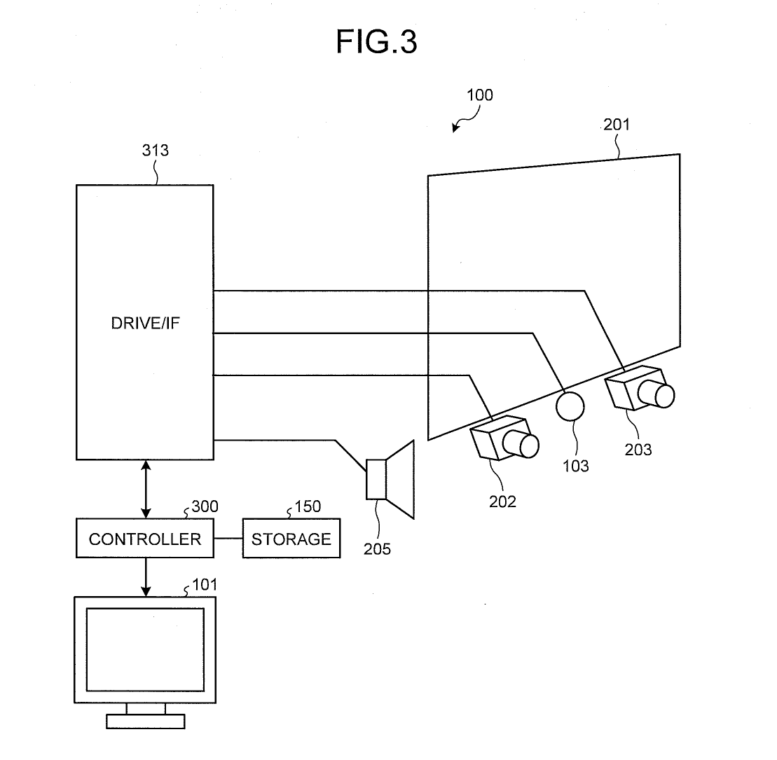

FIG. 3 is a diagram illustrating an outline of functions of the diagnosis supporting device 100. FIG. 3 illustrates part of the configuration illustrated in FIG. 1 and FIG. 2 and a configuration used for the drive or the like of the configuration. As illustrated in FIG. 3, the diagnosis supporting device 100 includes the right camera 202, the left camera 203, the LED light source 103, a speaker 205, a drive-and-interface (I/F) 313, a controller 300, a storage 150, and the display 101. Although FIG. 3 illustrates a display screen 201 in such a manner that position relation with the right camera 202 and the left camera 203 is easy to understand, the display screen 201 is a screen displayed on the display 101. The driver and the IF may be integral or separate.

The speaker 205 functions as a voice output unit that outputs a voice for calling subject's attention or the like during calibration or the like.

The drive/IF 313 drives units included in the stereo camera 102. The drive/IF 313 serves as an interface between the units included in the stereo camera 102 and the controller 300.

The controller 300 is implemented by a computer or the like including, for example, a controller such as a central processing unit (CPU), storage devices such as a read only memory (ROM) and a random access memory (RAM), a communication I/F that connects to a network to perform communication, and a bus that connects the units to each other.

The storage 150 stores therein various types of information such as control programs, measurement results, and diagnosis support results. The storage 150, for example, stores therein images or the like to be displayed on the display 101. The display 101 displays various types of information such as images to be diagnosed.

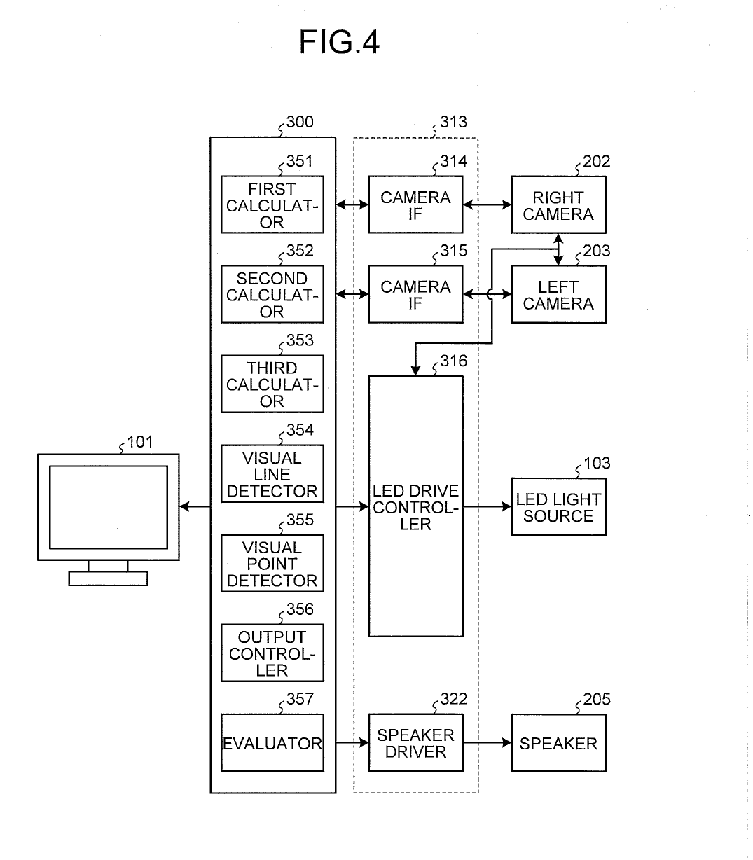

FIG. 4 is a block diagram illustrating an example of detailed functions of the units illustrated in FIG. 3. As illustrated in FIG. 4, the display 101 and the drive/IF 313 are connected to the controller 300. The drive/IF 313 includes camera IFs 314 and 315, an LED drive controller 316, and a speaker driver 322.

The right camera 202 and the left camera 203 are connected to the drive/IF 313 via the camera IFs 314 and 315, respectively. The drive/IF 313 drives the cameras, thereby imaging the subject.

The speaker driver 322 drives the speaker 205. The diagnosis supporting device 100 may include an interface (a printer IF) for connecting to a printer as a printing unit. The printer may be incorporated into the diagnosis supporting device 100.

The controller 300 controls the entire diagnosis supporting device 100. The controller 300 includes a first calculator 351, a second calculator 352, a third calculator 353, a visual line detector 354, a visual point detector 355, an output controller 356, and an evaluator 357. A visual line detection supporting device that detects a visual line only needs to include at least the first calculator 351, the second calculator 352, the third calculator 353, and the visual line detector 354.

Each of the components (the first calculator 351, the second calculator 352, the third calculator 353, the visual line detector 354, the visual point detector 355, the output controller 356, and the evaluator 357) included in the controller 300 may be implemented by software (a computer program), may be implemented by a hardware circuit, or may be implemented by using both the software and the hardware circuit.

When each of the components is implemented by the program, the program is recorded in a computer-readable recording medium such as a compact disc read only memory (CD-ROM), a flexible disk (FD), a compact disc recordable (CD-R), and a digital versatile disc (DVD) as an installable or executable file and is provided as a computer program product. The program may be stored in a computer connected to a network such as the Internet and provided by being downloaded via the network. The program may be provided or distributed via a network such as the Internet. The program may be embedded and provided in a ROM, for example.

The first calculator 351 calculates a position (a first position) of a pupil center indicating the center of a pupil from an image of an eyeball imaged by the stereo camera 102. The second calculator 352 calculates a position (a second position) of a corneal reflex center indicating the center of a corneal reflex from the taken image of the eyeball. The first calculator 351 and the second calculator 352 correspond to a position detector that detects the first position indicating the center of the pupil and the second position indicating the center of the corneal reflex.

The third calculator 353 calculates a corneal curvature center (a fourth position) from a line (a first line) connecting between the LED light source 103 and the corneal reflex center. The third calculator 353, for example, calculates a position that is on the line and the distance of which from the corneal reflex center is a certain value as the corneal curvature center. The certain value can be a value determined in advance from a general corneal curvature radius value or the like.

The corneal curvature radius value can have individual differences, and when the corneal curvature center is calculated using the value determined in advance, a large error may possibly occur. Given this situation, the third calculator 353 may calculate the corneal curvature center in consideration of the individual differences. In this case, the third calculator 353 first, using the pupil center and the corneal reflex center calculated when the subject is made to gaze at a target position (a third position), calculates a point of intersection between a line (a second line) connecting between the pupil center and the target position and a line (the first line) connecting between the corneal reflex center and the LED light source 103. The third calculator 353 then calculates a distance (a first distance) between the pupil center and the calculated point of intersection and stores the distance, for example, in the storage 150.

The target position may be a position that is determined in advance and the three-dimensional world coordinate values of which can be calculated. The central position (the point of origin of the three-dimensional world coordinates) of the display screen 201, for example, can be the target position. In this case, the output controller 356, for example, displays an image (a target image) at which the subject is made to gaze at the target position (center) of the display screen 201. With this configuration, the subject can be made to gaze at the target position.

The target image may be any image so long as it is an image at which the subject can be made to gaze. Examples of the target image include an image the display manner such as brightness and color of which changes and an image the display manner of which is different from the other areas.

The target position is not limited to the center of the display screen 201 and may be any position. The center of the display screen 201 as the target position minimizes the distance to any end of the display screen 201. This arrangement can reduce a measurement error at the time of visual line detection, for example.

The processing up to the calculation of the distance is performed in advance, for example, before starting actual visual line detection. At the time of the actual visual line detection, the third calculator 353 calculates a position that is on the line connecting between the LED light source 103 and the corneal reflex center and the distance from the pupil center of which is the distance calculated in advance as the corneal curvature center. The third calculator 353 corresponds to a calculator that calculates a corneal curvature center (the fourth position) from the position of the LED light source 103, a certain position (the third position) indicating the target image on the display 101, the position of the pupil center, and the position of the corneal reflex center.

The visual line detector 354 detects a visual line of the subject from the pupil center and the corneal curvature center. The visual line detector 354, for example, detects a direction from the corneal curvature center toward the pupil center as a visual line direction of the subject.

The visual point detector 355 detects a visual point of the subject using the detected visual line direction. The visual point detector 355, for example, detects a visual point (a gazing point), which is a point at which the subject gazes on the display screen 201. The visual point detector 355 detects a point of intersection between a visual line vector represented in a three-dimensional world coordinate system as in, for example, FIG. 2 and an XY-plane as the gazing point of the subject.

The output controller 356 controls output of various types of information for the display 101 and the speaker 205. The output controller 356, for example, outputs the target image at the target position on the display 101. The output controller 356 controls output of a diagnostic image, evaluation results by the evaluator 357, or the like to the display 101.

The diagnostic image may be an image appropriate for evaluation processing based on a visual line (visual point) detection result. When a developmental disorder is diagnosed, for example, a diagnostic image containing an image (a geometrical pattern video or the like) that subjects of the developmental disorder like and other images (portrait videos or the like) may be used.

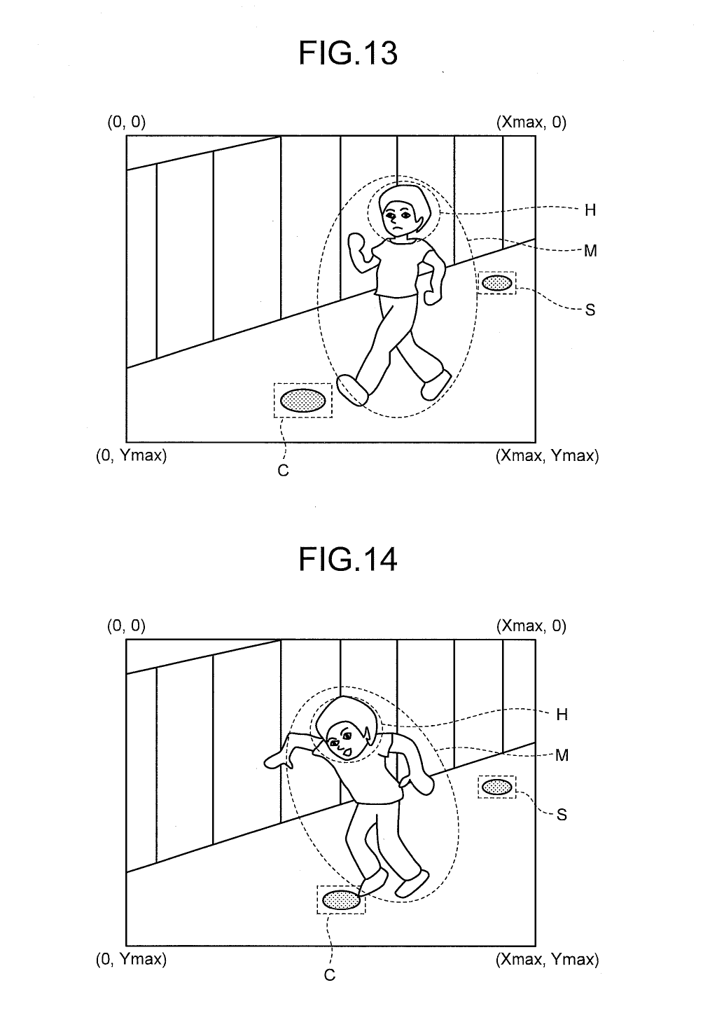

The evaluator 357 performs evaluation processing based on the diagnostic image and the gazing point detected by the visual point detector 355. When a developmental disorder is diagnosed, for example, the evaluator 357 analyzes the diagnostic image and the gazing point and evaluates whether the image that subjects of the developmental disorder like has been gazed at. The evaluator 357, for example, calculates an evaluation value based on the position of the gazing point by the subject when such diagnostic images as illustrated in FIG. 13 and FIG. 16 described below are displayed. A specific example of a method for calculating the evaluation value will be described below. The evaluator 357 only needs to calculate the evaluation value based on the diagnostic image and the gazing point, and the method for calculating the evaluation value is not limited to the present embodiment.

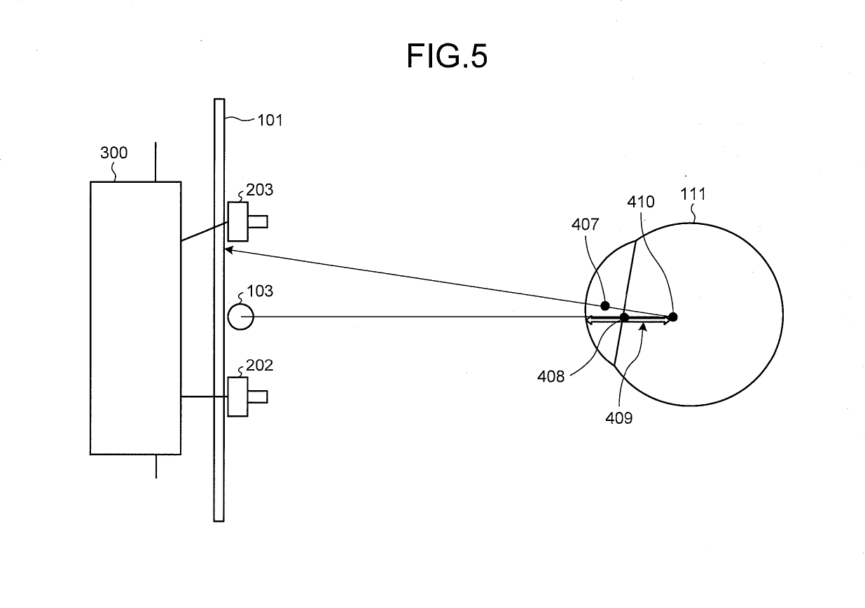

FIG. 5 is a diagram illustrating an outline of processing performed by the diagnosis supporting device 100 of the present embodiment. The components described in FIG. 1 to FIG. 4 are attached with the same signs, and descriptions thereof are omitted.

A pupil center 407 and a corneal reflex center 408 represent the center of a pupil and the center of a corneal reflex point, respectively, detected when the LED light source 103 is turned on. A corneal curvature radius 409 represents the distance from a corneal surface to a corneal curvature center 410.

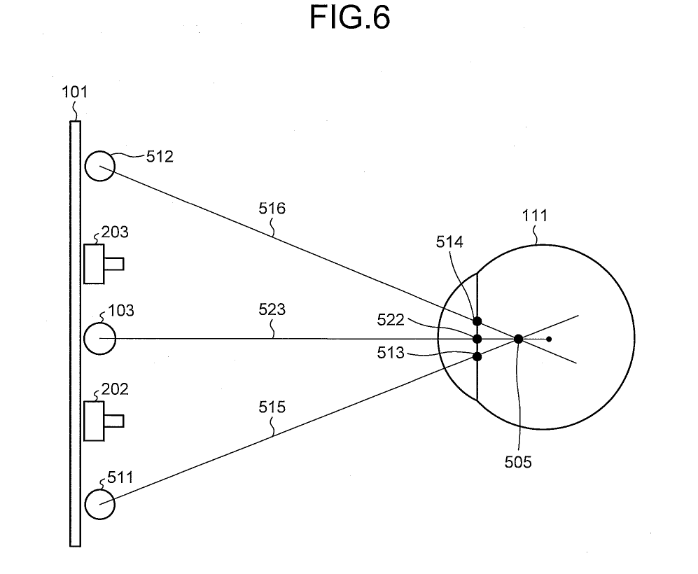

FIG. 6 is an illustrative diagram illustrating a difference between a method (hereinafter, referred to as a method A) using two light sources (the illuminator) and the present embodiment using one light source (the illuminator). The components described in FIG. 1 to FIG. 4 are attached with the same signs, and descriptions thereof are omitted.

The method A uses two LED light sources 511 and 512 in place of the LED light source 103. The method A calculates a point of intersection between a line 515 connecting between a corneal reflex center 513 illuminated by the LED light source 511 and the LED light source 511 and a line 516 connecting between a corneal reflex center 514 illuminated by the LED light source 512 and the LED light source 512. This point of intersection is a corneal curvature center 505.

In contrast, considered in the present embodiment is a line 523 connecting between a corneal reflex center 522 illuminated by the LED light source 103 and the LED light source 103. The line 523 passes through the corneal curvature center 505. It is known that the curvature radius of the cornea has nearly a constant value with less influence of individual differences. From this fact, the corneal curvature center illuminated by the LED light source 103 is present on the line 523 and can be calculated using the general curvature radius value.

However, when a visual point is calculated using the position of the corneal curvature center determined using the general curvature radius value, a visual point position deviates from the original position due to individual differences in the eyeball, which may fail to perform accurate visual point detection.

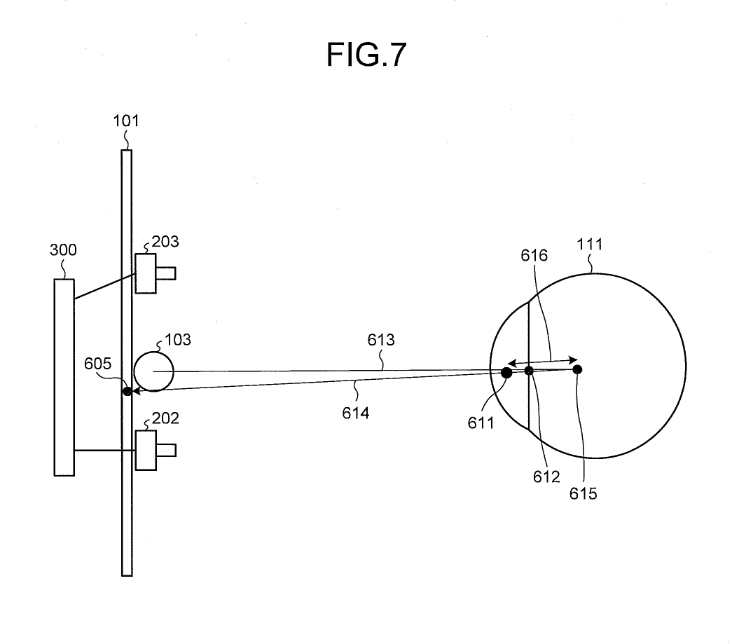

FIG. 7 is a diagram for illustrating calculation processing for calculating the corneal curvature center position and the distance between a pupil center position and a corneal curvature center position before performing visual point (visual line) detection. The components described in FIG. 1 to FIG. 4 are attached with the same signs, and descriptions thereof are omitted.

A target position 605 is a position at which the target image or the like is displayed at one point on the display 101 and at which the subject is made to gaze. In the present embodiment, the target position 605 is the central position of the screen of the display 101. A line 613 is a line connecting between the LED light source 103 and a corneal reflex center 612. A line 614 is a line connecting between the target position 605 (the gazing point) at which the subject gazes and a pupil center 611. A corneal curvature center 615 is a point of intersection between the line 613 and the line 614. The third calculator 353 calculates and stores therein a distance 616 between the pupil center 611 and the corneal curvature center 615.

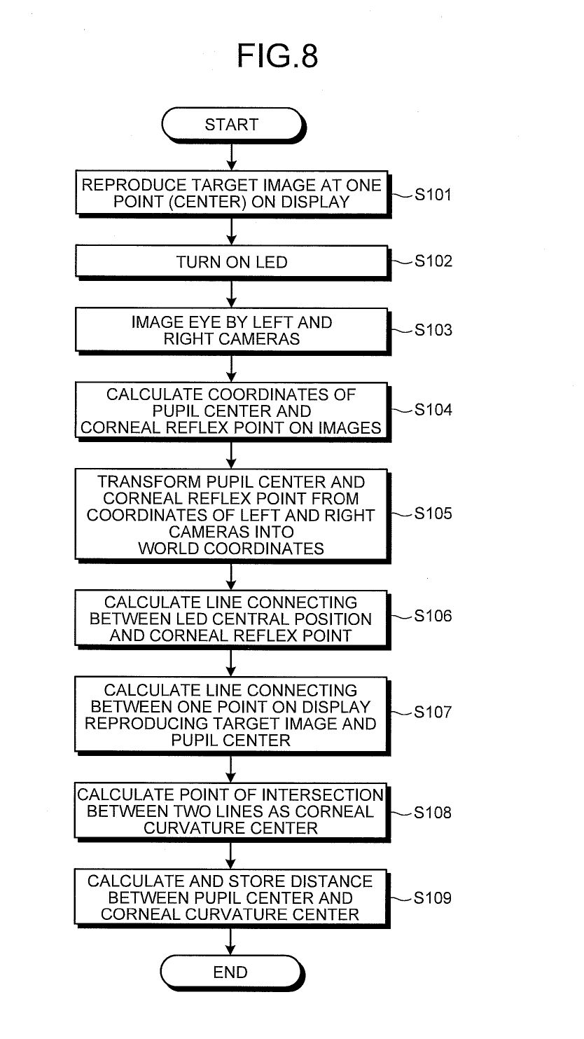

FIG. 8 is a flowchart illustrating an example of calculation processing of the present embodiment.

First, the output controller 356 reproduces the target image at one point on the screen of the display 101 (Step S101) and causes the subject to gaze at the one point. Next, the controller 300 turns on the LED light source 103 toward an eye of the subject using the LED drive controller 316 (Step S102). The controller 300 images the eye of the subject by the left and right cameras (the right camera 202 and the left camera 203) (Step S103).

By the emission from the LED light source 103, a pupil part is detected as a dark part (a dark pupil). A virtual image of a corneal reflex occurs as a reflection of the LED emission, and the corneal reflex point (corneal reflex center) is detected as a bright part. Specifically, the first calculator 351 detects the pupil part from the taken image and calculates coordinates indicating the position of the pupil center. The first calculator 351, for example, detects an area of certain brightness or less containing the darkest part in a certain area containing the eye as the pupil part and detects an area of certain brightness or more containing the brightest part as the corneal reflection. The second calculator 352 detects a corneal reflex part from the taken image and calculates coordinates indicating the position of the corneal reflex center. The first calculator 351 and the second calculator 352 calculate the coordinate values for the respective two images acquired by the left and right cameras (Step S104).

The right and left cameras are subjected to camera calibration by a method of stereo calibration in advance in order to acquire three-dimensional world coordinates, and transformation parameters are calculated. The method of stereo calibration may be any of conventionally used methods such as the method using the camera calibration theory by Tsai.

The first calculator 351 and the second calculator 352, using the transformation parameters, transforms the coordinates of the left and right camera into three-dimensional world coordinates of the pupil center and the corneal reflex center (Step S105). The third calculator 353 obtains a line connecting between the determined world coordinates of the corneal reflex center and the world coordinates of a center position of the LED light source 103 (Step S106). Next, the third calculator 353 calculates a line connecting between the world coordinates of the center of the target image displayed at one point on the screen of the display 101 and the world coordinates of the pupil center (Step S107). The third calculator 353 obtains a point of intersection between the line calculated at Step S106 and the line calculated at Step S107 and determines the point of intersection to be the corneal curvature center (Step S108). The third calculator 353 calculates the distance between the pupil center and the corneal curvature center in this situation and stores the distance in the storage 150 or the like (Step S109). The stored distance is used for calculating the corneal curvature center at the time of subsequent visual point (visual line) detection.

The distance between the pupil center and the corneal curvature center when the subject gazes at the one point on the display 101 in the calculation processing is maintained constant within the range of detecting the visual point within the display 101. The distance between the pupil center and the corneal curvature center may be determined from the average of total values calculated during the reproduction of the target image or determined from the average of several values among the values calculated during the reproduction.

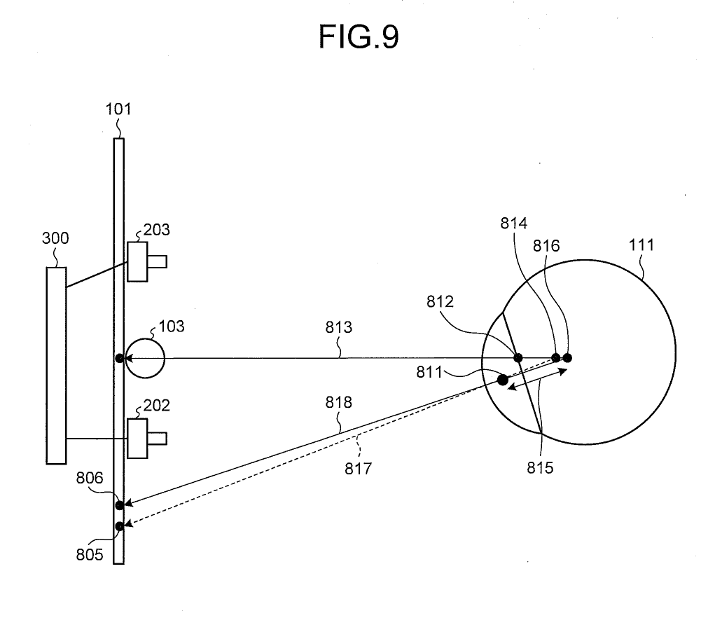

FIG. 9 is a diagram illustrating a method for calculating a corrected position of the corneal curvature center using the distance between the pupil center and the corneal curvature center determined in advance at the time of visual point detection. A gazing point 805 represents a gazing point determined from the corneal curvature center calculated using the general curvature radius value. A gazing point 806 represents a gazing point determined from the corneal curvature center calculated using the distance determined in advance.

A pupil center 811 and a corneal reflex center 812 indicate the position of the pupil center and the position of the corneal curvature center, respectively, calculated at the time of visual point detection. A line 813 is a line connecting between the LED light source 103 and the corneal reflex center 812. A corneal curvature center 814 is the position of the corneal curvature center calculated from the general curvature radius value. A distance 815 is the distance between the pupil center and the corneal curvature center calculated by the advance calculation processing. A corneal curvature center 816 is the position of the corneal curvature center calculated using the distance determined in advance. The corneal curvature center 816 is determined by the corneal curvature center that is present on the line 813 and the distance between the pupil center and the corneal curvature center that is the distance 815. With this determination, a visual line 817 calculated when the general curvature radius value is used is corrected to a visual line 818. The gazing point on the screen of the display 101 is corrected from the gazing point 805 to the gazing point 806.

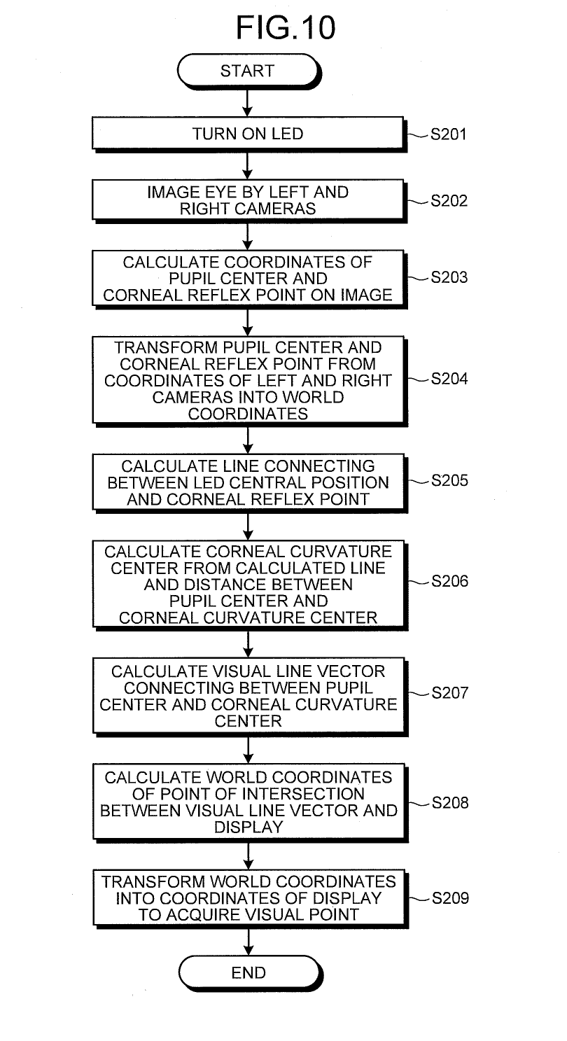

FIG. 10 is a flowchart illustrating an example of visual line detection processing of the present embodiment. The visual line detection processing of FIG. 10 can be performed as processing to detect a visual line in diagnosis processing using the diagnostic image. In the diagnosis processing, in addition to the steps of FIG. 10, processing to display the diagnostic image, evaluation processing by the evaluator 357 using the detection result of the gazing point, and the like are performed.

Steps S201 to S205 are similar to Steps S102 to S106 of FIG. 8, and descriptions thereof are omitted.

The third calculator 353 calculates a position which is on the line calculated at Step S205 and that the distance from the pupil center is equal to the distance determined by the calculation processing in advance, as the corneal curvature center (Step S206).

The visual line detector 354 determines a vector (visual line vector) connecting between the pupil center and the corneal curvature center (Step S207). The vector indicates the visual line direction that the subject is seeing. The visual point detector 355 calculates three-dimensional world coordinate values of a point of intersection between the visual line direction and the screen of the display 101 (Step S208). The values are coordinate values representing the one point on the display 101 at which the subject gazes with the world coordinates. The visual point detector 355 transforms the determined three-dimensional world coordinate values into coordinate values (x, y) represented by a two-dimensional coordinate system of the display 101 (Step S209). With this transformation, the visual point (gazing point) on the display 101 at which the subject gazes can be calculated.

First Modification

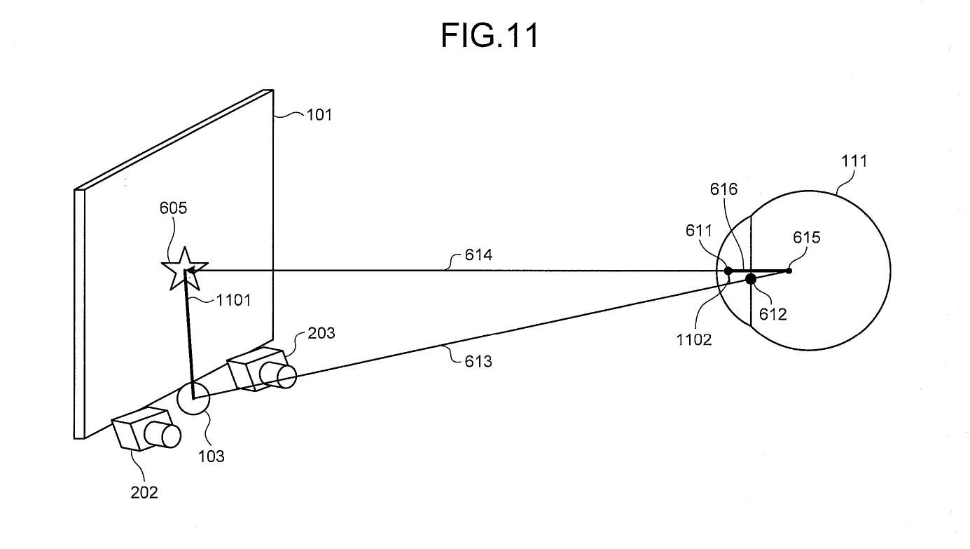

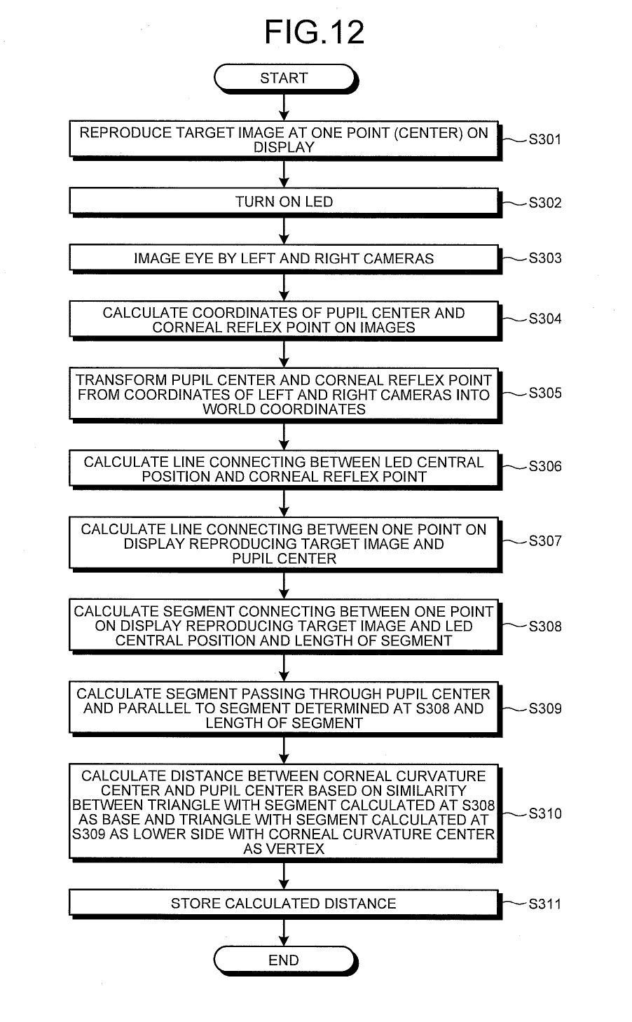

The calculation processing to calculate the distance between the pupil center position and the corneal curvature center position is not limited to the method described in FIG. 7 and FIG. 8. The following describes another example of the calculation processing with reference to FIG. 11 and FIG. 12.

FIG. 11 is a diagram for illustrating calculation processing of the present modification. The components described in FIG. 1 to FIG. 4 and FIG. 7 are attached with the same signs, and descriptions thereof are omitted.

A segment 1101 is a segment (a first segment) connecting between the target position 605 and the LED light source 103. A segment 1102 is a segment (a second segment) that is parallel to the segment 1101 and connects between the pupil center 611 and the line 613. The present modification calculates and stores the distance 616 between the pupil center 611 and the corneal curvature center 615 using the segment 1101 and the segment 1102 as follows.

FIG. 12 is a flowchart illustrating an example of the calculation processing of the present modification.

Steps S301 to S307 are similar to Steps S101 to S107 of FIG. 8, and descriptions thereof are omitted.

The third calculator 353 calculates a segment (the segment 1101 in FIG. 11) connecting between the center of the target image displayed at the one point of the screen of the display 101 and the center of the LED light source 103 and calculates the length (defined as L1101) of the calculated segment (Step S308).

The third calculator 353 calculates a segment (the segment 1102 in FIG. 11) that passes through the pupil center 611 and is parallel to the segment calculated at Step S308 and calculates the length (defined as L1102) of the calculated segment (Step S309).

The third calculator 353, based on a similarity relation between a triangle with the corneal curvature center 615 as a vertex and with the segment calculated at Step S308 as a base and a triangle with the corneal curvature center 615 as a vertex and with the segment calculated at Step S309 as a base, calculates the distance 616 between the pupil center 611 and the corneal curvature center 615 (Step S310). The third calculator 353, for example, calculates the distance 616 so that the ratio of the length of the segment 1102 to the length of the segment 1101 is equal to the ratio of the distance 616 to the distance between the target position 605 and the corneal curvature center 615.

The distance 616 can be calculated by the following equation (1), where L614 is the distance from the target position 605 to the pupil center 611.

Distance 616=(L614×L1102)/(L1101−L1102) (1)

The third calculator 353 stores the calculated distance 616 in the storage 150 or the like (Step S311). The stored distance is used for calculating the corneal curvature center at the time of subsequent visual point (visual line) detection.

The following describes details of diagnosis support processing. The present embodiment uses an image representing a cause of a certain event and the event as the diagnostic image. Measuring a dwell time at a gazing point for an area set in the diagnostic image can support diagnosis. This configuration can support diagnosis such as what point developmentally disabled people gaze at to obtain information in order to understand a causal relation, whether they are unable to understand the causal relation despite gazing at, or the like. In other words, diagnosis support with higher precision than conventional ones is provided.

FIG. 13 to FIG. 16 are diagrams illustrating examples of the diagnostic image for use in the present embodiment. Each of the diagnostic images of FIG. 13 to FIG. 16 is an example of an image indicating one scene contained in a continuous moving image. The continuous moving image may be a moving image containing a cut assignment (image switching or the like) in the middle thereof.

FIG. 13 is an image indicating a scene in which a person is walking on a road in front of a fence. A plurality of stones is at his feet. Areas are set for the image. The example of FIG. 13 sets an area M containing a person, an area H containing a head, an area C containing a stone (an example of a first object) as a cause by which the person falls down, and an area S containing a stone that is irrelevant to the event in which the person (an example of a second object) falls down. Each of the images of FIG. 13 to FIG. 16 has a coordinate system with the upper-left of the image as the point of origin (0, 0) and with the lower-right coordinates as (Xmax, Ymax).

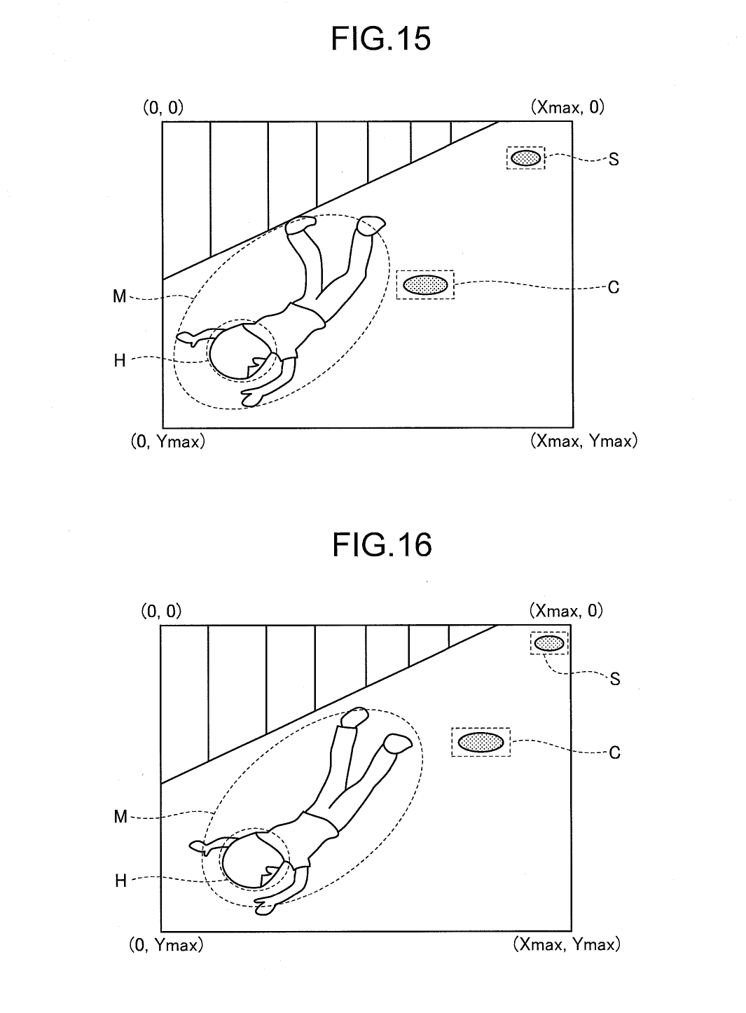

FIG. 14 is an image at the moment when the person stumbles over the stone within the area C and is about to fall down. FIG. 15 is an image at the moment when the person stumbles over the stone within the area C and falls down. FIG. 16 is an image indicating a scene after the person stumbles over the stone within the area C and falls down. Areas similar to those of FIG. 13 are set also for the respective images of FIG. 14 to FIG. 16.

The present embodiment uses, as one of the diagnostic images, a still image obtained by capturing a part of the moving image before an event occurs or a still image equivalent to the still image and a still image obtained by capturing a part of the moving image after the event occurs or a still image equivalent to the still image.

In the examples of FIG. 13 to FIG. 16, the event is “falling down of the person.” A still image 1 (FIG. 13) obtained by capturing a part of the moving image before the event and a still image 2 (FIG. 16) obtained by capturing a part of the moving image after the event are used as the diagnostic image. The number of the still images is not limited to two, and three or more still images may be used.

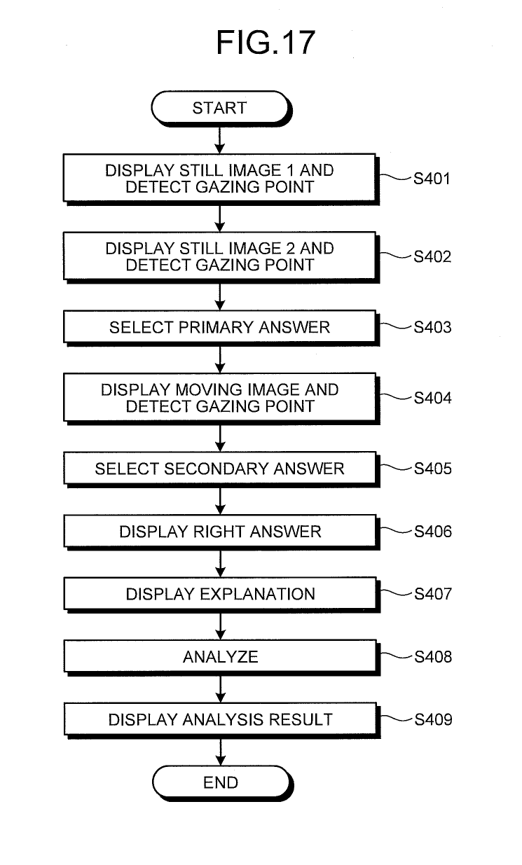

FIG. 17 is a flowchart illustrating an example of the diagnosis support processing when such diagnostic images are used.

First, the output controller 356 displays the still image 1 on the display 101. The subject sees the displayed still image 1. In this situation, the visual point detector 355 detects the gazing point (Step S401).

Next, the output controller 356 displays the still image 2 on the display 101. The subject sees the displayed still image 2. In this situation, the visual point detector 355 detects the gazing point (Step S402). Advancement from Step S401 to Step S402 may be performed in accordance with the pressing of a “proceed to next button” (not illustrated) or the like by the subject or an operator. The display may be continuously advanced without any instruction by the subject or the operator.

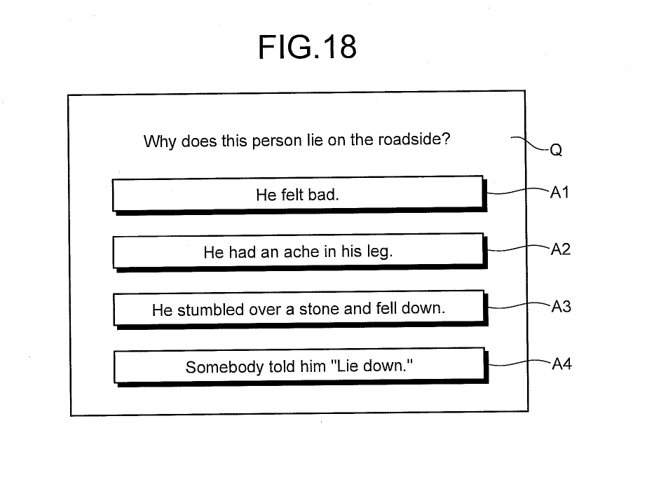

Next, the evaluator 357 receives a selection of a primary answer by the subject (Step S403). FIG. 18 is a diagram illustrating an example of a selection screen for selecting the primary answer. The primary answer is an answer selected after displaying the diagnostic images (the still image 1 and the still image 2). In the example of FIG. 18, the primary answer to a question Q is selected from among answer options A1 to A4. The answer options may include a noun indicating at least one of the cause and the event. In FIG. 18, for example, the option A3 to be a right answer contains a noun “stone” representing the cause and a verb “fell down” representing the event. Subjects having a developmental disorder have difficulty in determining a causal relation and often select any other than A3. On a probabilistic basis, the right answer may be selected even without understanding the causal relation. Only one question may therefore fail to support diagnosis with high precision. Consequently, inspections may be performed similarly for many kinds of videos. This operation can further improve the accuracy of diagnosis support.

The evaluator 357 acquires position information indicating a position touched by the subject or the operator from the display 101 configured as, for example, a touch panel and receives the selection of an option corresponding to the position information. The evaluator 357 may receive the primary answer designated using an input device (a keyboard or the like, not illustrated) by the subject or the operator. The selection of the primary answer is not limited to the method using the selection screen as illustrated in FIG. 18. For example, a method may be used that causes the operator to explain the question and the answer options orally and causes the subject to select the primary answer orally.

Returning back to FIG. 17, the output controller 356, for example, displays a moving image (a continuous moving image corresponding to the diagnostic images in FIG. 13 to FIG. 16) corresponding to the diagnostic images (the still image 1 and the still image 2) on the display 101. In this situation, the visual point detector 355 detects the gazing point (Step S404).

Details of gazing point detection processing at Step S401, Step S402, and Step S404 will be described below.

The evaluator 357 then receives a selection of a secondary answer by the subject (Step S405). The secondary answer is an answer selected after displaying the moving image corresponding to the diagnostic images. The selection of the secondary answer may be performed by, for example, a similar method to the selection of the primary answer. Options of the secondary answer may be the same as the options of the primary answer or different therefrom. The secondary answer is selected in order to enable the determination (the primary answer) after seeing the still image 1 and the still image 2 and the determination (the secondary answer) after seeing the continuous moving image subsequently to be compared with each other.

Next, the output controller 356 displays the right answer to the question on the display 101 (Step S406). The output controller 356 displays an explanation on the display 101 (Step S407).

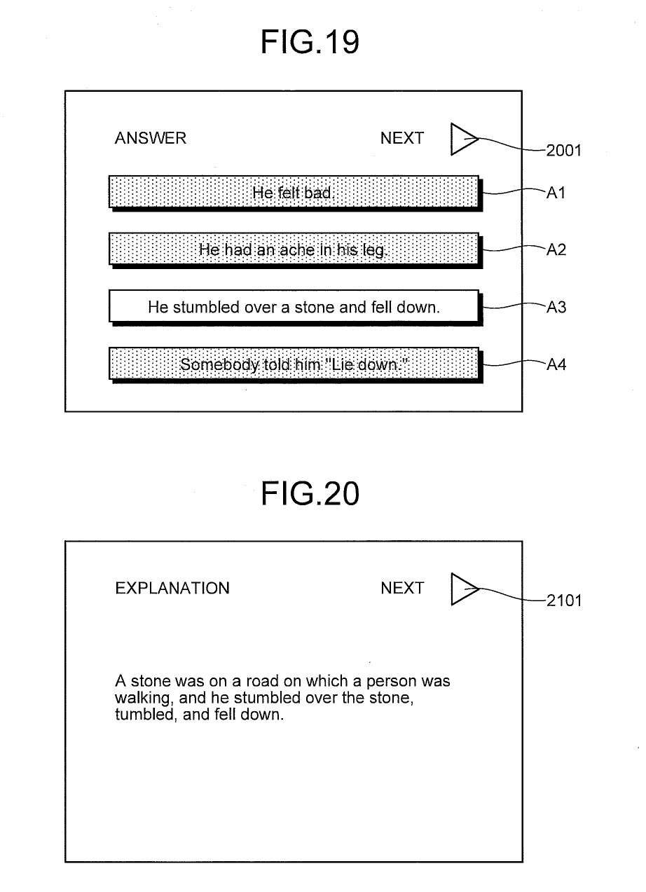

FIG. 19 is a diagram illustrating an example of a right answer screen for displaying the right answer. In the example of FIG. 19, the option A3 indicating the right answer is displayed in a display manner different from those of the other options. The method for indicating the right answer is not limited to this. If the options of the primary answer and the options of the secondary answer are different from each other, all the options may be displayed to highlight the option of the right answer.

On this right answer screen, for example, if a next button 2001 is pressed, an explanation screen for displaying an explanation is displayed. FIG. 20 is a diagram illustrating an example of the explanation screen. The explanation screen is displayed, thereby enabling the subject to understand the causal relation of the event indicated in the diagnostic images or the like. If a next button 2101 is pressed on the explanation screen, the display of the explanation screen is ended.

Returning back to FIG. 17, the evaluator 357 performs analysis processing based on data of the detected gazing point (Step S408). Details of the analysis processing will be described below. Finally, the output controller 356 displays an analysis result on the display 101 or the like (Step S409).

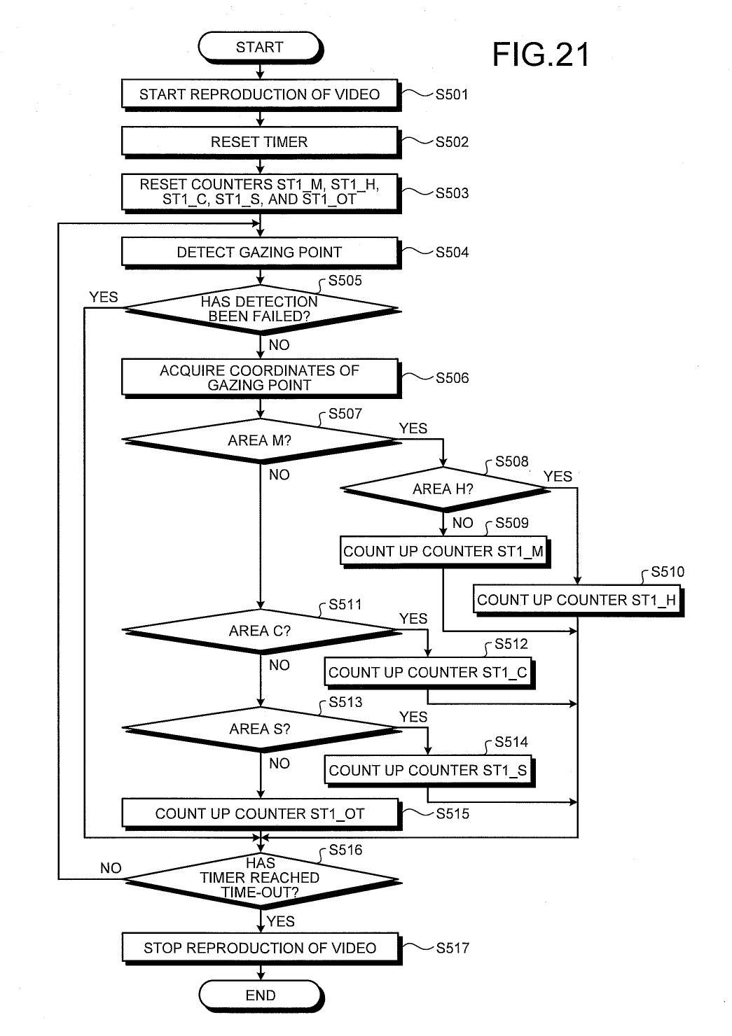

The following describes the details of the gazing point detection processing. FIG. 21 is a flowchart illustrating an example of the gazing point detection processing. FIG. 21 illustrates the gazing point detection processing after displaying the still image 1 at Step S401 of FIG. 17 as an example, and the pieces of gazing point detection processing at Step S402 (after displaying the still image 2) and Step S404 (after displaying the moving image) of FIG. 17 can also be achieved by a similar procedure.

First, the output controller 356 starts reproduction (display) of the diagnostic image (the still image 1) (Step S501). Next, the output controller 356 resets a timer for measuring a reproduction time (Step S502). Next, the visual point detector 355 resets counters (counters ST1_M, ST1_H, ST1_C, ST1_S, and ST1_OT) that count up at the time of gazing at the respective areas (Step S503).

The counters ST1_M, ST1_H, ST1_C, ST1_S, and ST1_OT are counters when the still image 1 (ST1) is displayed. The respective counters correspond to the following areas. By counting up the respective counters, dwell times representing times during which the gazing point is detected within the respective corresponding areas can be measured.

The counter ST1_M: the area M

The counter ST1_H: the area H

The counter ST1_C: the area C

The counter ST1_S: the area S

The counter ST1_OT: an area other than the above areas

Next, the visual point detector 355 detects the gazing point of the subject (Step S504). The visual point detector 355, for example, can detect the gazing point by the procedure described in FIG. 10. The visual point detector 355 determines whether detection of the gazing point has been failed (Step S505). When the images of the pupil and the corneal reflex cannot be obtained owing to a blink or the like, for example, the gazing point detection is failed. It may also be determined to be failed when the gazing point is absent within the display 101 (when the subject sees any other than the display 101).

If the detection of the gazing point has been failed (Yes at Step S505), the process advances to Step S516. If detection of the gazing point has been succeeded (No at Step S505), the visual point detector 355 acquires coordinates of the gazing point (gazing point coordinates) (Step S506).

The visual point detector 355 determines whether the acquired gazing point coordinates are present within the area M (around the person) (Step S507). If the acquired gazing point coordinates are present within the area M (Yes at Step S507), the visual point detector 355 further determines whether the acquired gazing point coordinates are present within the area H (around the head) (Step S508). If the acquired gazing point coordinates are present within the area H (Yes at Step S508), the visual point detector 355 increments (counts up) the counter ST1_H (Step S510), and the process proceeds to Step S516. If the acquired gazing point coordinates are absent within the area H (No at Step S508), the visual point detector 355 increments (counts up) the counter ST1_M (Step S509), and the process proceeds to Step S516.

If the acquired gazing point coordinates are absent within the area M (No at Step S507), the visual point detector 355 determines whether the acquired gazing point coordinates are present within the area C (around the object as the cause of the causal relation) (Step S511). If the acquired gazing point coordinates are present within the area C (Yes at Step S511), the visual point detector 355 increments (counts up) the counter ST1_C (Step S512), and the process proceeds to Step S516.

If the acquired gazing point coordinates are absent within the area C (No at Step S511), the visual point detector 355 determines whether the acquired gazing point coordinates are present within the area S (around the object that is not the cause of the causal relation) (Step S513). If the acquired gazing point coordinates are present within the area S (Yes at Step S513), the visual point detector 355 increments (counts up) the counter ST1_S (Step S514), and the process proceeds to Step S516.

If the acquired gazing point coordinates are absent within the area S (No at Step S513), the gazing point is absent within any of the set areas, and the visual point detector 355 increments (counts up) the counter ST1_OT (Step S515).

Next, the output controller 356 checks whether the timer that manages the reproduction time of the video has reached a time-out (Step S516). If a certain time has not been elapsed, that is, if the timer has not reached a time-out (No at Step S516), the process returns to Step S504 to continue the measurement. If the timer has reached a time-out (Yes at Step S516), the output controller 356 stops reproduction of the video (Step S517).

The gazing point detection processing when the still image 2 (ST2) at Step S402 is displayed can use a similar procedure to FIG. 21 by replacing the counters as follows:

The counter ST1_M→a counter ST2_M

The counter ST1_H→a counter ST2_H

The counter ST1_C→a counter ST2_C

The counter ST1_S→a counter ST2_S

The counter ST1_OT→a counter ST2_OT

The gazing point detection processing when the moving image (MOV) at Step S404 is displayed can use a similar procedure to FIG. 21 by replacing the counters as follows:

The counter ST1_M→a counter MOV_M

The counter ST1_H→a counter MOV_H

The counter ST1_C→a counter MOV_C

The counter ST1_S→a counter MOV_S

The counter ST1_OT→a counter MOV_OT

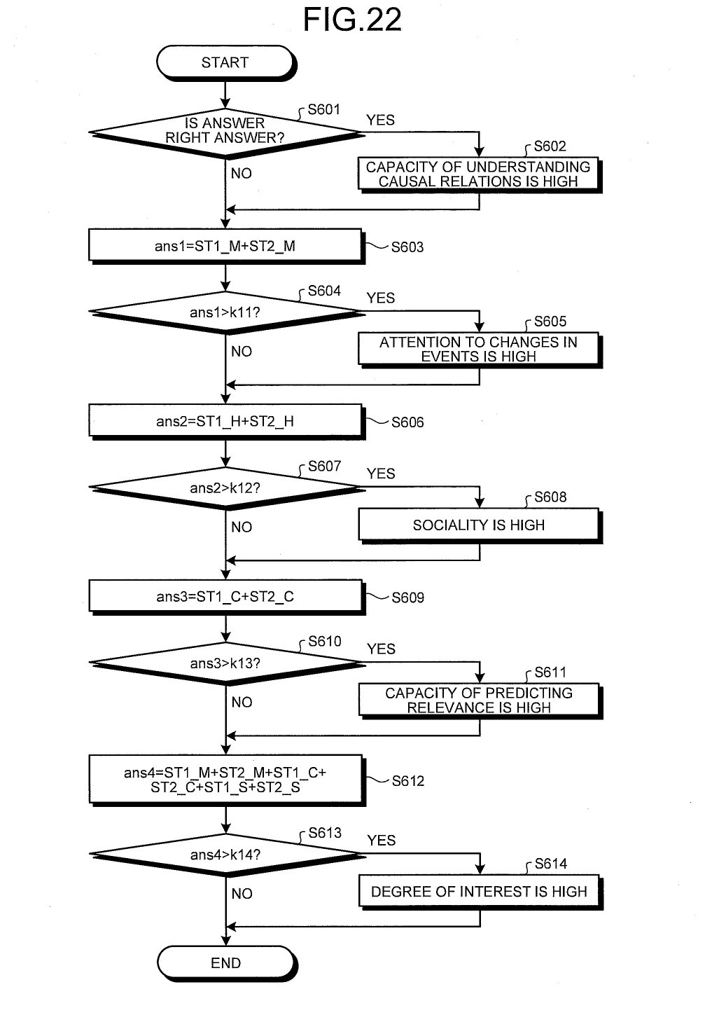

The following describes the details of the analysis processing. FIG. 22 is a flowchart illustrating an example of the analysis processing. The analysis processing and the evaluation value described below are examples and not limited to them. The evaluation value, for example, may be changed in accordance with the displayed diagnostic image.

First, the evaluator 357 determines whether the selected primary answer is the right answer (Step S601). If the selected primary answer is the right answer (Yes at Step S601), the evaluator 357 calculates an evaluation value indicating that capacity of understanding causal relations is high (Step S602).

If the primary answer is not the right answer (No at Step S601), or after Step S602, the evaluator 357 calculates ans1=ST1_M+ST2_M (Step S603). ST1_M represents a value of the counter ST1_M, for example. The following may similarly represent a value of a counter X as simply “X.”

Next, the evaluator 357 determines whether ans1 is larger than a threshold k11 (Step S604). If ans1 is larger than the threshold k11 (Yes at Step S604), the evaluator 357 calculates an evaluation value indicating that the degree of attention is high against changes in events (Step S605). This is because ans1 indicates a degree to which the gazing point is contained within the area M containing the person. The evaluation value may be binary values indicating that the degree of attention toward changes in events is high or low or multiple values varying in accordance with, for example, the magnitude of ans1.

If ans1 is equal to or less than the threshold k11 (No at Step S604), or after Step S605, the evaluator 357 calculates ans2=ST1_H+ST2_H (Step S606). The evaluator 357 then determines whether ans2 is larger than a threshold k12 (Step S607). If ans2 is larger than the threshold k12 (Yes at Step S607), the evaluator 357 calculates an evaluation value indicating that the degree of attention toward the head containing one's face is high and that the development of sociality is high (Step S608). This is because ans2 indicates a degree to which the gazing point is contained within the area H containing the head.

If ans2 is equal to or less than the threshold k12 (No at Step S607), or after Step S608, the evaluator 357 calculates ans3=ST1_C+ST2_C (Step S609). The evaluator 357 then determines whether ans3 is larger than a threshold k13 (Step S610). If ans3 is larger than the threshold k13 (Yes at S610), the evaluator 357 calculates an evaluation value indicating that capacity of predicting relevance is high and that attention to the object related to the causal relation is paid (Step S611). This is because ans3 indicates a degree to which the gazing point is contained within the area C containing the stone as the cause by which the person falls down.

If ans3 is equal to or less than the threshold k13 (No at Step S610), or after Step S611, the evaluator 357 calculates ans4=ST1_M+ST2_M+ST1_C+ST2_C+ST1_S+ST2_S (Step S612). The evaluator 357 then determines whether ans4 is larger than a threshold k14 (Step S613). If ans4 is larger than the threshold k14 (Yes at Step S613), the evaluator 357 calculates an evaluation value, which indicates that the degree of interest toward various objects and events is high (Step S614). This is because ans4 indicates a degree to which the gazing point is contained within the areas (the area M, the area C, and the area S) containing the objects such as the person and the stone.

In the case that ans4 is equal to or less than the threshold k14 (No at Step S613), or after Step S614, the analysis processing ends. Similarly to ans1, ans2, ans3, and ans4 may be binary values or multiple values.

Subjects having a developmental disorder often have difficulty in understanding causal relations. It is desirable to change a method of care and education depending on whether a causal relation cannot be understood even though a subject gazes at a cause of the causal relation and incorporates its information into the brain or the causal relation cannot be understood because the subject does not try to see the cause of the causal relation, and information itself does not reach the brain. In particular, when a case has neither the evaluation value indicating that capacity of understanding causal relations is high (Step S602), the evaluation value indicating that the development of sociality is high (Step S608), nor the evaluation value indicating that capacity of predicting relevance is high (Step S611), the case has a possibility of a developmental disorder.

The diagnosis supporting device of the present embodiment, when images (the still image 1 and the still image 2, for example) before and after an event are displayed, measures what part a subject sees. With this configuration, diagnosis can be supported with high precision even about whether causal relations can be understood or the like. With an analyzed (diagnosed) result as reference, a policy of care and education can be determined.

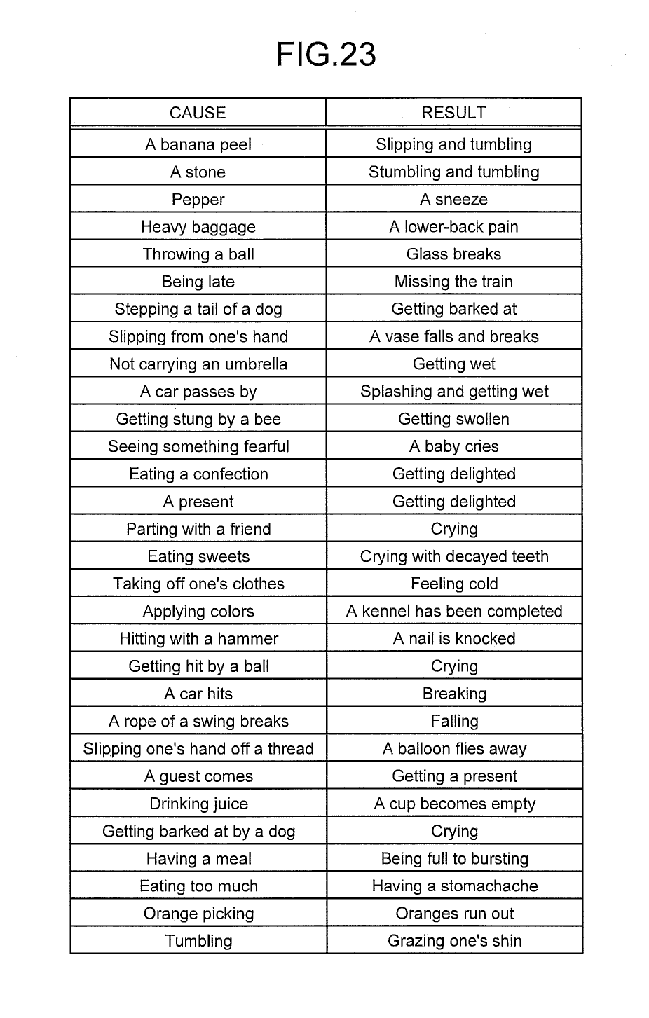

FIG. 23 is a diagram illustrating examples of the causal relation. It is illustrated that results described on the right column are produced in response to causes described on the left column. In place of the still image 1 and the still image 2, a still image indicating any cause described in FIG. 23 and a still image indicating a result corresponding to the cause may be used. Other than the examples of FIG. 23, various diagnostic images indicating causes and results can be used. The diagnostic images (two still images or the like) indicating causal relations as illustrated in FIG. 23, for example, may be displayed a plurality of times, and evaluation results for a plurality of diagnostic images may be integrated. For example, the values of the respective counters may not be reset each time the diagnostic images are displayed, and addition of the values of the counters for all the diagnostic images may be continued. In this case, the respective thresholds used in the analysis processing in FIG. 22, for example, may be changed in accordance with the number, type, or the like of the used diagnostic images. With this configuration, the accuracy of evaluation can further be increased.

As illustrated in FIG. 22, in principle, diagnosis is supported based on the gazing point when the images (the still image 1 and the still image 2) before and after the event are displayed. Display of the moving image, detection of the gazing point when the moving image is displayed, and display of the explanation (Step S404 to Step S407 in FIG. 17) are provided in order to tell the subject the right answer and enable evaluation on whether the subject has understood causal relations by seeing the moving image, or the like. These pieces of processing (Step S404 to Step S407 in FIG. 17) can be omitted for the purpose of diagnosis support alone, for example.

When the evaluation value (Step S611) indicating that capacity of predicting relevance is high is calculated, for example, diagnosis about understanding of causal relations can be supported. In this case, there is no need to display the right answer screen or to receive selection of the primary answer. This is because what is only needed is the detection result of the gazing point when the diagnostic image is displayed, for calculating the evaluation value as in Step S611.

In FIG. 22, the evaluation values were each independently calculated. Two or more of the conditions of FIG. 22 may be combined to determine an evaluation value. For example, If the primary answer is the right answer (Yes at Step S601) and if ans3 is larger than the threshold k13 (Yes at Step S610), the evaluation value indicating that capacity of predicting relevance is high may be calculated.

The diagnosis support processing of FIG. 17 includes display of the explanation image (moving image) illustrating the causal relation (Step S404), display of the right answer (Step S406), and display of the explanation (Step S407). Consequently, diagnosis can be supported, and in addition, support for training can also be achieved. Repeating the processing of FIG. 17 for the same diagnostic image or a plurality of different diagnostic images, for example, can provide more effective support for training.

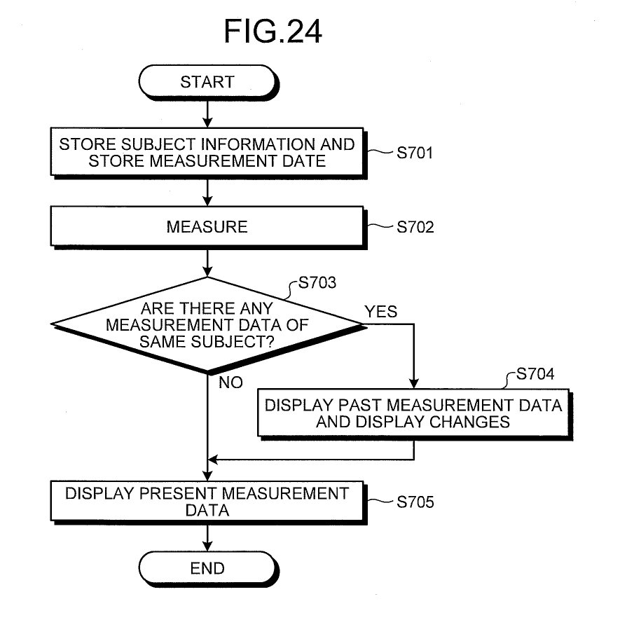

FIG. 24 is a flowchart illustrating an example of verification processing that verifies and displays effects of care and education.

First, the evaluator 357 stores subject information such as the name of a subject and a measurement date, for example, in the storage 150 before measurement (Step S701). Next, the diagnosis support processing (measurement) as illustrated in FIG. 17 is performed (Step S702). The evaluator 357 then determines whether past measurement data of the same subject is stored (Step S703). The measurement data includes, for example, values (dwell times) of the respective counters, ans1 to ans4 calculated from the values of the respective counters, and a part of or the entire evaluation values.

If the past measurement data is stored (Yes at Step S703), the output controller 356 displays information indicating the past measurement data and a change in the present measurement data relative to the past measurement data on the display 101 (Step S704).

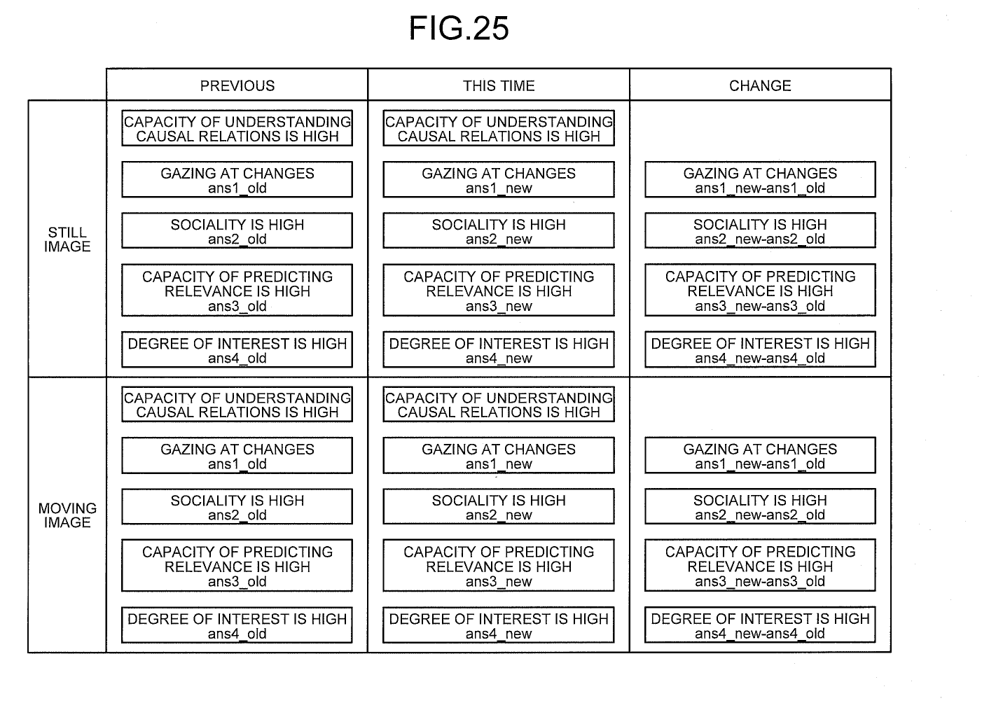

FIG. 25 is a diagram for illustrating an example of a method for determining changes in the measurement data. FIG. 25 illustrates examples in which changes in the present measurement data relative to the previous measurement data are separately determined for a still image and a moving image.

Concerning the evaluation value of “capacity of understanding causal relations is high,” for example, it is illustrated that there is no change. ansn_old (n is 1 to 4) indicates values of the previous measurement data. ansn_new (n is 1 to 4) indicates values of the present measurement data. As illustrated in FIG. 25, changes in the measurement data can be determined by, for example, the difference between ansn_new and ansn_old.

The output controller 356 displays information (values indicating the difference) indicating thus determined changes in the measurement data, for example, on the display 101. The output controller 356 may output the measurement data and the information indicating changes to another device (an external communication device connected via a network, a printer, or the like) in place of the display 101.

Returning back to FIG. 24, in the case that the past measurement data is not stored (No at Step S703), or after the display processing at Step S704, the output controller 356 displays the present measurement data on the display 101 or the like (Step S705). If the past measurement data is present, the output controller 356 may simultaneously display the previous measurement data, the present measurement data, and the information indicating changes.

Thus, training by seeing the diagnostic image several times can increase the capacity of understanding causal relations and enables effects by the training to be checked.

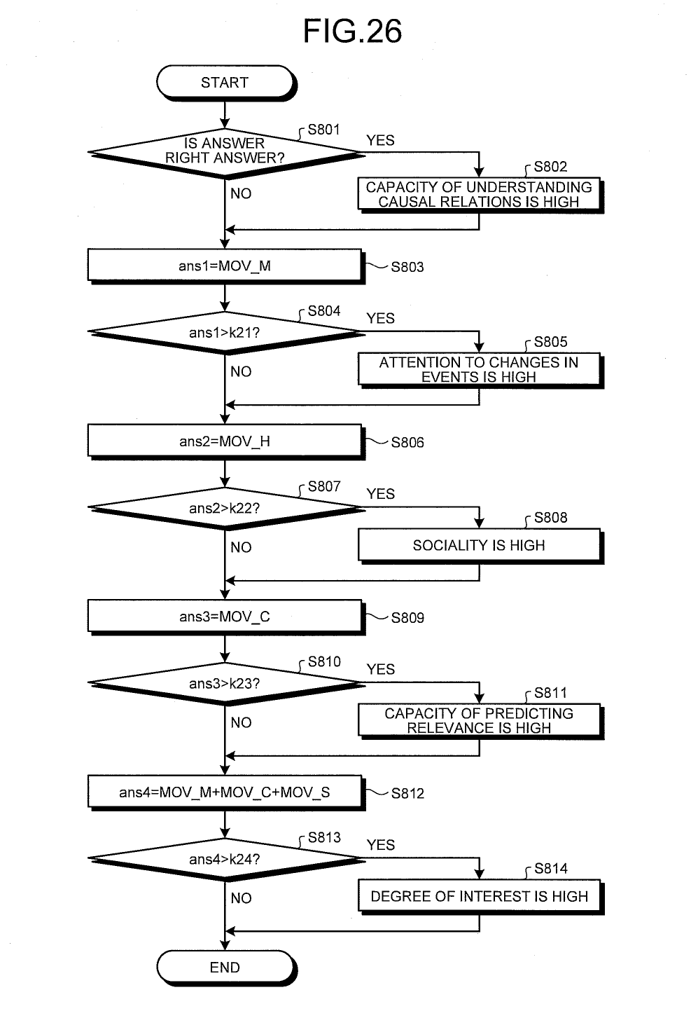

The analysis processing may be performed also when a moving image is displayed. FIG. 26 is a flowchart illustrating an example of the analysis processing when the moving image is displayed. In comparison with FIG. 22, in FIG. 26, the method for calculating ans1 to ans4 and the thresholds are changed as follows. The other procedure of processing is the same as that of FIG. 22, and a description thereof is omitted.

ans1=MOV_M

ans2=MOV_H

ans3=MOV_C

ans4=MOV_M+MOV_C+MOV_S

k11→k21

k12→k22

k13→k23

k14→k24

The analysis processing as illustrated in FIG. 26 enables the subject to be evaluated on an increase of understanding by seeing the moving image. For example, the evaluation result of the moving image may further be added to the evaluation result of FIG. 22. With this configuration, the accuracy of evaluation can further be increased.

In order to support the determination of the policy of care and education, methods of training (a policy of care and education) to be recommended may be displayed on the display 101 or the like in accordance with a diagnostic result. For example, the evaluator 357 may compare the measurement data with a threshold for policy determination determined in advance, and the output controller 356 may display different methods of training for a case of being lower than the threshold and a case of being the threshold or more. The output controller 356 may display different methods of training in accordance with a combination of values of different pieces of measurement data (the evaluation value indicating capacity of understanding causal relations is high and the evaluation value indicating capacity of predicting relevance is high, for example) or the like. The method of training may be a method of training using the present diagnosis supporting device 100, a method of training using illustrations and photographs, and any other method of training.

Although the above example uses the moving image containing the diagnostic images (the still image 1 and the still image 2) used at the time of diagnosis as the explanation image, the explanation image is not limited to such a moving image. Any image can be used so long as it is an image that represents a causal relation between a cause and an event and serves as support for training. For example, the explanation image containing one or more still images different from the diagnostic images may be used.

Second Modification

The above embodiment describes an example in which the diagnosis supporting device that supports diagnosis of a developmental disorder or the like is used also as a training supporting device. Any device that can display, for example, an explanation image, a right answer, an explanation, or the like other than the diagnosis supporting device can be used as the training supporting device. The present modification describes an example in which a portable terminal such as a tablet, a smartphone, and a notebook personal computer (PC) is used as the training supporting device. Other than that, an information processing device such as an ordinary personal computer can be used as the training supporting device.

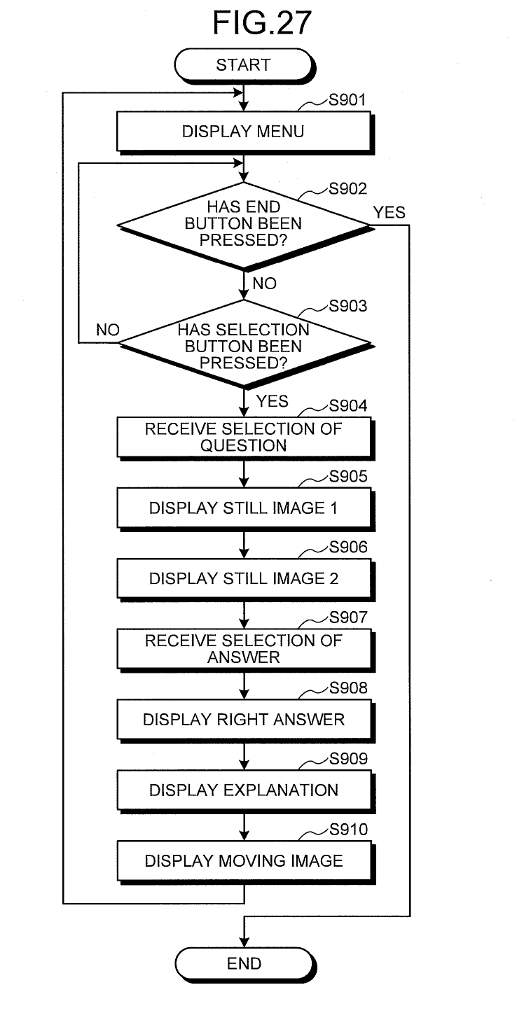

The gazing point detection processing performed at Step S401, Step S402, and Step S404 of the diagnosis support processing of FIG. 17, for example, is used for calculating the evaluation values used for diagnosis support mainly for a developmental disorder. When the object is training support, therefore, it is not necessary to perform the gazing point detection processing. The following describes an example of training support processing that does not include the gazing point detection processing. FIG. 27 is a flowchart illustrating an example of the training support processing of the present modification.

When a program for training support is started, for example, the output controller 356 displays a menu screen (Step S901).

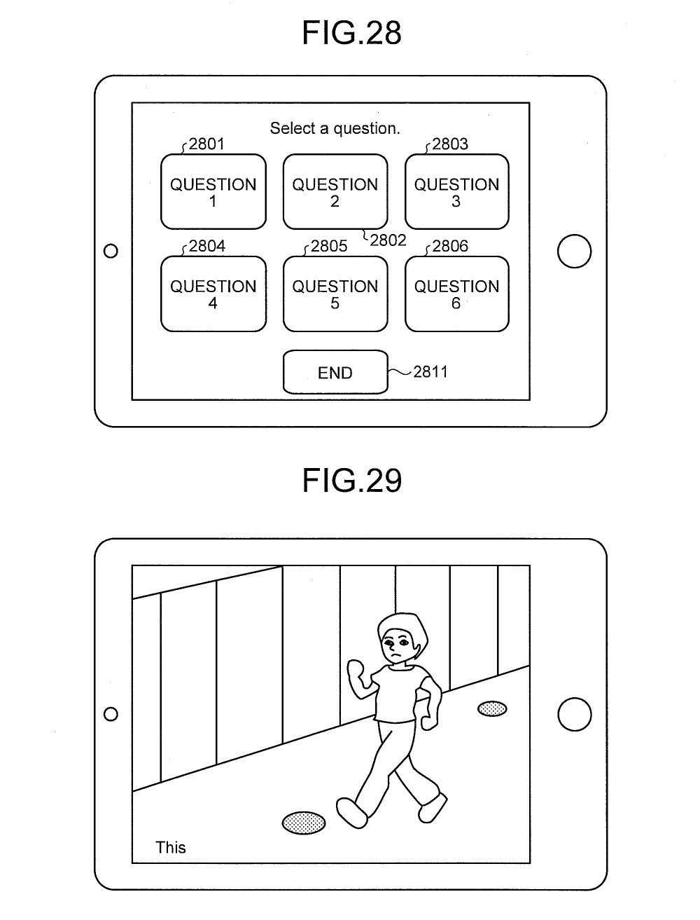

FIG. 28 is a diagram illustrating an example of the menu screen of Modification 2. As illustrated in FIG. 28, the menu screen contains selection buttons 2801 to 2806 for selecting a question and an end button 2811. If any of the selection buttons 2801 to 2806 is pressed, an image of a corresponding question is displayed. FIG. 28 illustrates an example in which six questions (Question 1 to Question 6) can be selected. The number of the questions and the method for selecting a question are not limited to the example of FIG. 28. If the end button 2811 is pressed, the program ends.

Returning back to FIG. 27, the output controller 356 determines whether the end button 2811 has been pressed (Step S902). If the end button 2811 has been pressed (Yes at Step S902), the output controller 356 ends the training support processing.

If the end button 2811 has not been pressed (No at Step S902), the output controller 356 determines whether any of the selection buttons 2801 to 2806 has been pressed (Step S903). If any of the selection buttons 2801 to 2806 has not been pressed (No at Step S903), the process returns to Step S902 to repeat the processing.

If any of the selection buttons 2801 to 2806 has been pressed (Yes at Step S903), the output controller 356 receives selection of a question corresponding to the pressed button among the selection buttons 2801 to 2806 (Step S904). The output controller 356 displays the still image 1 indicating the cause of the event among the images corresponding to the received question (Step S905). Suppose that, for example, a user (trainee) has pressed the selection button 2801 of FIG. 28 to select Question 1. FIG. 29 is a diagram illustrating an example of the still image 1 displayed in this situation. FIG. 29 illustrates an example of the still image containing an object (stone) as a cause.

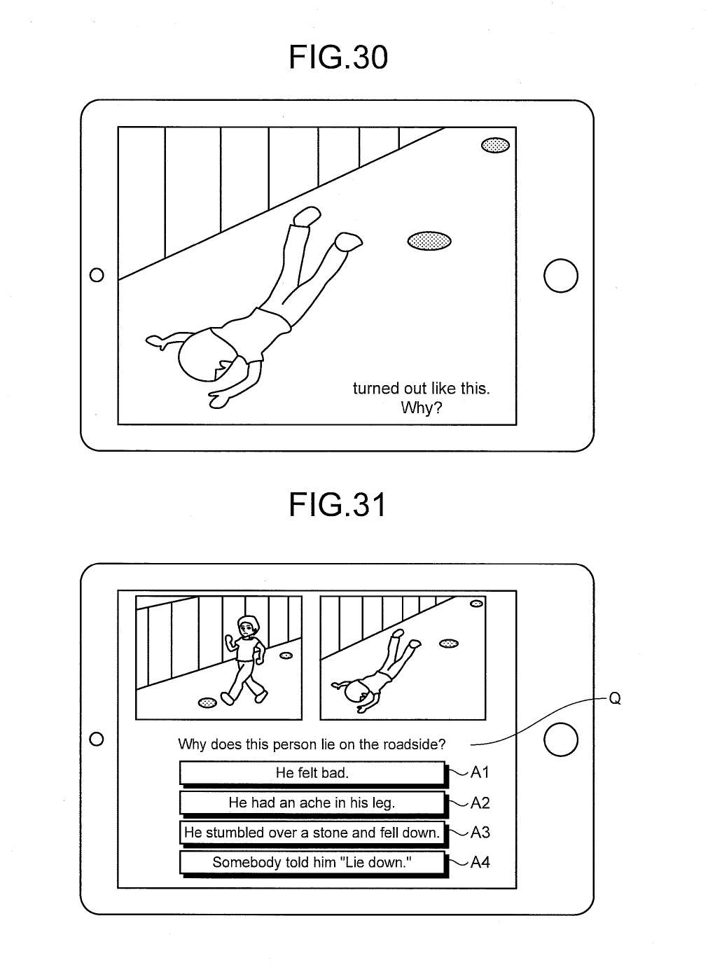

Returning back to FIG. 27, the output controller 356 displays the still image 1 for a certain time (10 seconds, for example) and then displays the still image 2 indicating an event for a certain time (10 seconds, for example) (Step S906). The display times of the respective still images may be the same or different from each other. FIG. 30 is a diagram illustrating an example of the still image 2 displayed in this situation. FIG. 30 is an example of the still image indicating a result (falling down) caused by the object (stone) as the cause.

Next, the output controller 356 receives selection of an answer (Step S907). FIG. 31 is a diagram illustrating an example of a selection screen for selecting the answer. FIG. 31 illustrates an example of the selection screen containing, together with the two still images (the still image 1 and the still image 2), a question Q and answer options A1 to A4. The user selects an answer to the question Q from among the answer options A1 to A4. The output controller 356 receives the answer thus selected by the user.

Returning back to FIG. 27, the output controller 356 displays a right answer to the question on the display 101 (Step S908). The output controller 356 displays an explanation on the display 101 (Step S909).

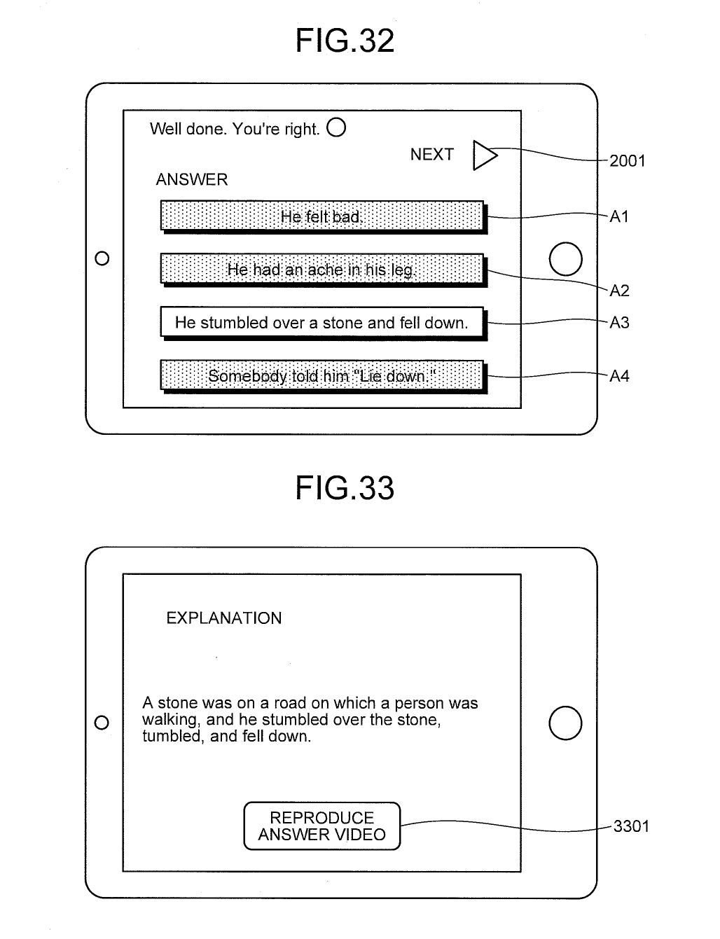

FIG. 32 is a diagram illustrating an example of a right answer screen for displaying the right answer. The example of FIG. 32 displays, together with a result of the answer (“Well done. You're right. 0”), the option A3 indicating the right answer in a display manner (not grayed out) different from those of the other options. If a next button 2001 is pressed on this right answer screen, for example, an explanation screen for displaying an explanation is displayed. FIG. 33 is a diagram illustrating an example of the explanation screen. The explanation screen is displayed, thereby enabling the subject to understand the causal relation of the event or the like displayed in the diagnostic image. If a button 3301 is pressed on the explanation screen, a reproduction screen that reproduces a moving image (a reproduction video) is displayed.

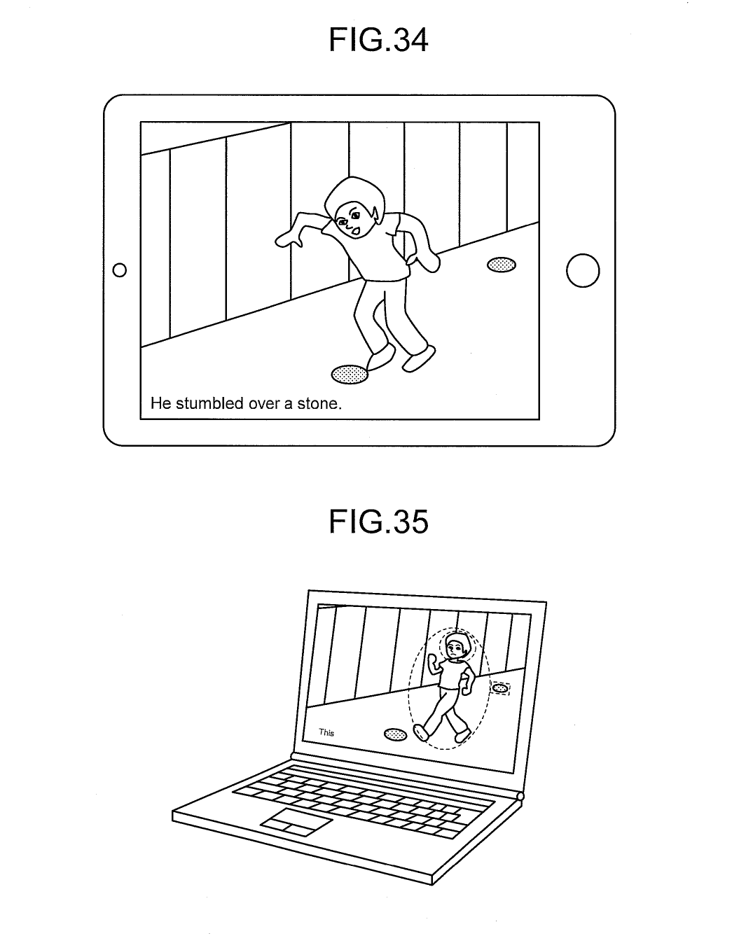

If the button 3301 is pressed on the explanation screen, the output controller 356 displays the reproduction screen on the display 101 (Step S910). The reproduction screen displays a moving image containing, for example, a process from the still image 1 to the still image 2. FIG. 34 is a diagram illustrating an example of the reproduction screen. This reproduction screen is an image at a certain point of time of the reproduced moving image and illustrates an example in which the image containing an explanation (“He stumbled over a stone”) is displayed. Such a moving image is displayed, thereby enabling the user to further deepen understanding of the causal relation of the event or the like.

When the display of the reproduction screen ends, the process returns to the menu display (Step S901).

Such processing enables training even by an device such as a tablet, which does not incorporate a gazing point detector and is less expensive. It is noted that a doctor or the like cannot perform evaluation or guidance based on a gazing point during training.

FIG. 35 is a diagram illustrating an example of implementing a training supporting device by a notebook PC. FIG. 35 illustrates an example in which the still image 1 corresponding to FIG. 29 is displayed on a display (corresponding to the display 101) of the notebook PC.

If the answer by the user is the right answer, points or the like may be given for each right answer. With this configuration, the user is motivated to perform training, and training can be supported more effectively.

As described above, the present embodiment produces the following advantageous effects, for example.

(1) By causing a subject to view images for training multiple times, the subject is enabled to understand a causal relation effectively.
(2) Effects of training can be measured.
(3) Whether a subject gazes at an object related to a causal relation can be known.
(4) Points and directions of care and education can be set.
(5) Self-analysis is enabled.
(6) Development of sociality can also be checked.
(7) Without the need to arrange light sources (the illuminator) at two sites, visual line detection is enabled using a light source arranged at one site.
(8) Owing to the light source arranged at one site, the device can be compact, and cost reduction can also be achieved.

The diagnosis supporting device, the diagnosis supporting method, and the computer-readable recording medium according to the present embodiment produce the advantageous effect of increasing the accuracy of diagnosis.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A diagnosis supporting device comprising:
a display;
an imaging unit that images a subject;
a visual line detector that detects a visual line direction of the subject from an image imaged by the imaging unit;
a visual point detector that detects a visual point of the subject in a display area of the display based on the visual line direction;
an output controller that displays a diagnostic image representing a cause of a certain event and the event on the display; and
an evaluator that calculates an evaluation value of the subject based on the visual point detected by the visual point detector when the diagnostic image is displayed.

2. The diagnosis supporting device according to claim 1, wherein
the output controller further outputs a question about the event, and
the evaluator calculates the evaluation value of the subject based on the visual point and an answer to the question.

3. The diagnosis supporting device according to claim 1, wherein the evaluator calculates the evaluation value based on at least one of a first dwell time indicating a time during which the visual point is detected in a first area containing a first object as the cause among areas contained in the diagnostic image and a second dwell time indicating a time during which the visual point is detected in a second area containing a second object as an object in which the event occurs among the areas contained in the diagnostic image.

4. The diagnosis supporting device according to claim 3, wherein the evaluator calculates the evaluation value further based on a third dwell time indicating a time during which the visual point is detected in an area other than the first area and the second area among the areas contained in the diagnostic image.

5. The diagnosis supporting device according to claim 1, wherein
the output controller displays a plurality of diagnostic images, and
the evaluator calculates the evaluation value of the subject based on the visual point detected by the visual point detector when the plurality of diagnostic images are displayed.

6. The diagnosis supporting device according to claim 1, further comprising:
an illuminator including a light source that emits light;
a position detector that detects a first position indicating center of a pupil and a second position indicating center of a corneal reflex from an image of an eyeball of the subject to which light is applied by the illuminator and that is imaged by the imaging unit; and
a calculator that calculates a fourth position indicating a corneal curvature center based on a position of the light source, a third position on the display, the first position, and the second position, wherein
the visual line detector detects a visual line of the subject based on the first position and the fourth position.

7. A diagnosis supporting method comprising:
detecting, from an image imaged by an imaging unit that images a subject, a visual line direction of the subject;
detecting a visual point of the subject in a display area of a display based on the visual line direction;
displaying a diagnostic image representing a cause of a certain event and the event on the display; and
calculating an evaluation value of the subject based on the visual point detected at the detecting a visual point of the subject when the diagnostic image is displayed.

8. A non-transitory computer-readable recording medium that therein stores a computer program causing a computer to execute a diagnosis supporting method, the method comprising:
displaying a diagnostic image representing a cause of a certain event and the event on a display; and
displaying an explanation image representing a causal relation between the cause and the event on the display.
